SEQUENCE LISTING (8 sequences)

SEQ ID NO: 1 - protein, 22 aa, Artificial
  CsgB peptide
LRQGGSKLLAVVAQEGSSNRAK

SEQ ID NO: 2 - protein, 20 aa, Artificial
  CsgB peptide
GTQKTAIVVQRQSQMAIRVT

SEQ ID NO: 3 - protein, 5 aa, Artificial
  CsgB peptide
AIVVQ

SEQ ID NO: 4 - protein, 6 aa, Artificial
  CsgB peptide
LAVVAQ

SEQ ID NO: 5 - protein, 16 aa, Artificial
  CsgB peptide
GGSKLLAVVAQEGSSN

SEQ ID NO: 6 - protein, 9 aa, Artificial
  CsgB peptide
KLLAVVAQE

SEQ ID NO: 7 - protein, 8 aa, Artificial
  CsgB peptide
KTAIVVQR

SEQ ID NO: 8 - protein, 17 aa, Artificial
  CsgB peptide
TQKTAIVVQRQSQMAIR

(19) United States
(12) Patent Application Publication
(10) Pub. No.: US 2012/0190566 A1
(43) Pub. Date: Jul. 26, 2012

(54) COMPOSITIONS AND METHODS FOR INHIBITING BIOFILMS

(76) Inventors: Susan Lindquist, Chestnut Hill, MA (US); Rajaraman Krishnan, Quincy, MA (US)

(21) Appl. No.: 13/259,746

(22) PCT Filed: Mar. 25, 2010
(86) PCT No.: PCT/US2010/028703

§ 371 Date: Apr. 5, 2012

Related U.S. Application Data
(60) Provisional application No. 61/163,238, filed on Mar. 25, 2009.

(51) Int. Cl. C40B 30/04; C07K 14/00; C07K 14/195; C40B 40/10; C07K 14/26; C07K 14/255; C07K 14/25; G01N 33/566; C07K 2/00; C07K 14/245

(52) U.S. Cl. 506/9; 530/300; 436/501; 506/18

(57) ABSTRACT

Using arrays of peptides derived from E. coli CsgB, peptides that seed formation of curli fibers are identified. The arrays, peptides, methods for identification thereof, and compositions and methods relating thereto, are provided.

### RELATED APPLICATION(S)

This application claims the benefit of U.S. Provisional Application No. 61/163,238 filed on Mar. 25, 2009. The entire teachings of the above application are incorporated herein by reference.

### BACKGROUND

To establish successful infection, bacteria often encase themselves into a complex, polymeric biofilm that aids adhesion and significantly reduces their susceptibility to host defenses and to a broad spectrum of antimicrobial agents. Patients suffering from biofilm associated chronic infections, such as periodontal disease, endocarditis, otitis media (ear infections) and osteomyelitis, frequently experience cycles of acute exacerbation and remission that often results in treatment failure. Medical device-related infections associated with biofilms that are formed in catheter tubing, coronary stents, joint prostheses, intraocular lens and other implanted devices frequently require surgical removal of the device, despite appropriate therapy. Compositions and methods for combating biofilms are urgently needed.

### SUMMARY

The present invention relates in part to compositions and methods for identifying protein domains that nucleate assembly of protein aggregates comprised of two or more different polypeptides. The invention further relates to compositions and methods for identifying agents that modulate, e.g., inhibit or disrupt, formation or maintenance of protein aggregates comprised of two or more different polypeptides, e.g., protein aggregates in which a first polypeptide seeds formation of an aggregate comprised at least in part of a second polypeptide. The invention also relates to methods of using agents that modulate, e.g., inhibit or disrupt, formation or maintenance of protein aggregates comprised of two or more different polypeptides, e.g., protein aggregates in which a first polypeptide seeds formation of an aggregate comprised at least in part of a second polypeptide. In certain embodiments the invention relates to amyloids that are components of bacterial biofilms, peptides that nucleate formation of such amyloids, compositions and methods relating to such peptides, and methods of use thereof.

The invention also provides a collection comprising at least 10 different peptides, wherein the peptides are between 8 and 50 amino acid in length and have a sequence that comprises at least 8 and no more than 50 contiguous amino acids of a first amyloidogenic polypeptide, wherein the first amyloidogenic polypeptide is capable of nucleating formation of an amyloid that comprises a second amyloidogenic polypeptide. In some embodiments the first amyloidogenic polypeptide is a CsgB polypeptide and the second amyloidogenic polypeptide is a CsgA polypeptide.

The invention further provides a method of identifying an aggregation domain of a first amyloidogenic polypeptide comprising the steps of: (i) providing an array comprising a plurality of peptides, wherein the peptides are fragments of a first amyloidogenic polypeptide; (ii) contacting the array with a second amyloidogenic polypeptide; and (iii) identifying a peptide to which the second amyloidogenic polypeptide binds, thereby identifying an aggregation domain of the first amyloidogenic polypeptide. In some embodiments the first amyloidogenic polypeptide is a CsgB polypeptide and the second polypeptide is a CsgA polypeptide.

The invention further provides a method of identifying an agent for modulating amyloid formation or maintenance comprising: (i) providing a composition comprising: (a) a peptide that is between 8 and 50 amino acid in length and has a sequence that comprises at least 8 and no more than 50 contiguous amino acids of a first amyloidogenic polypeptide; (b) a second amyloidogenic polypeptide; and (c) a test agent, wherein the peptide is capable of binding to the second amyloidogenic polypeptide in the absence of the test agent; and (ii) identifying the test agent as an agent for modulating amyloid formation if presence of the test agent alters the extent or rate of binding of the peptide and the second amyloidogenic polypeptide. In certain embodiments the first amyloidogenic polypeptide is a CsgB polypeptide and the second amyloidogenic polypeptide is a CsgA polypeptide.

The invention further provides a method for identifying an agent for inhibiting amyloid formation or maintenance comprising: (i) providing a composition comprising: (a) a peptide that is between 8 and 50 amino acid in length and has a sequence that comprises at least 8 and no more than 50 contiguous amino acids of a first amyloidogenic polypeptide; (b) a second amyloidogenic polypeptide; and (c) a test agent, wherein the peptide is capable of binding to the second amyloidogenic polypeptide in the absence of the test agent; and (ii) identifying the test agent as an agent for inhibiting amyloid formation or maintenance if presence of the test agent reduces the extent or rate of binding of the peptide and the second amyloidogenic polypeptide. In certain embodiments the first amyloidogenic polypeptide is a CsgB polypeptide and the second amyloidogenic polypeptide is a CsgA polypeptide.

The invention further provides a peptide whose sequence comprises at least 5 and no more than 50 contiguous amino acids of the sequence of a CsgB polypeptide, wherein the peptide is capable of nucleating formation of an amyloid that comprises a CsgA polypeptide. In some embodiments the sequence of an inventive peptide comprises at least 5 and no more than 30 contiguous amino acids of the sequence of a CsgB polypeptide. In some embodiments the sequence of an inventive peptide comprises at least 8 and no more than 20 contiguous amino acids of the sequence of a CsgB polypeptide. Also provided are variants of such peptides, libraries comprising the peptides and/or peptide variants, compositions comprising the peptide(s) and/or peptide variant(s), and methods of using the peptides and peptide variants.

All references cited herein are incorporated by reference.

### BRIEF DESCRIPTION OF THE DRAWINGS

FIG. 1 is a pictorial representation of the curli fibers on the E. coli outer membrane. CsgA forms the major component of the curli complex, which is nucleated by a small number of membrane-bound CsgB molecules. This complex attaches to the cell surface together with polysaccharides.

FIG. 2A shows a schematic illustration of a peptide array experiment showing fiber assembly. Twenty-mer peptide sequences are spotted (e.g, immobilized via a reactive NHS-ester moiety) on a surface forming an array. Alexa-labeled proteins are incubated with the array and imaged using a genepix scanner. Peptide spots bound to proteins show intense fluorescence. FIG. 2B shows CsgA monomers labeled with alexa-647 incubated on an array containing CsgA and CsgB peptides. Soluble CsgA specifically recognizes CsgB peptides. FIG. 2C shows Congo red (CR) staining of bacterial cells. Wild type cells secrete curli proteins on their surface which in the presence of CR stain red. Cells lacking curli-related proteins such as CsgB (CsGB-), or containing mutations that prevent proper nucleation of curli proteins appear white/transparent. pΔAIVV cells lack the 4 residues within the nucleating sequence that are critical for nucleation.

FIG. 3A shows that various biofilm-forming proteins (SEQ ID NOS: 9-16) from E. coli, Shigella and Salmonella, in this case CsgA proteins, share ˜70% sequence identity, ˜84% sequence similarity (consensus sequence SEQ ID NO: 17). FIG. 3B shows a multiple sequence alignment of a number of E. coli, Shigella and Salmonella CsgA proteins (SEQ ID NOS: 18-79) together with accession numbers. FIG. 3C shows a multiple sequence alignment of E. coli, Shigella and Salmonella CsgB proteins (SEQ ID NOS: 80-142) together with accession numbers.

FIG. 4 shows inhibition of curli assembly in the presence of two classes of compounds that were originally shown to prevent amyloid assembly by different mechanisms. DAPH-12 (top) blocks tyrosine stacking, while Amphotericin B (bottom) binds to the edge strands and prevents amyloid assembly.

FIG. 5A provides a schematic representation of a ThT assay. CsgB nucleating peptides are immobilized and incubated with candidate peptides or compounds and purified CsgA, and then assayed for CsgA assembly. High fluorescent intensities (indicated by yellow color) indicate the assembly of curli amyloid. FIG. 5B shows a schematic representation of assembly of a polypeptide to form amyloid in the presence (red) and in the absence (blue) of an appropriate nucleating sequence as monitored by Thioflavin-T fluorescence.

FIG. 6A is a schematic showing a crystal violet staining of fibronectin coated plates. Bacterial cells are incubated with test compounds and plates washed to remove non-adherent cells at different time points. The attached cells are quantified by using the gram negative stain crystal violet. Blue wells represent presence of adhering bacteria. FIG. 6B is a high resolution EM showing E. coli encased in a biofilm containing curli (C) and polysaccharide (CA).

FIG. 7 shows results of an experiment in which CsgB peptides (SEQ ID NOS: 1 and 2) that nucleate assembly of CsgA were identified.

FIG. 8 shows an alignment of E. coli CsgA and CsgB (SEQ ID NOS: 143 and 144) used to identify CsgB peptides that nucleate CsgA assembly. CsgB peptides (SEQ ID NOS: 1 and 2) identified are shown in red. (Some sequences within CsgA are also shown in red.)

### DETAILED DESCRIPTION

The present invention relates in part to compositions and methods useful for identifying protein aggregation domains, i.e., domains that mediate assembly of higher ordered aggregates. The invention further relates to protein aggregation domains that promote biofilm formation. Aggregates of interest in certain embodiments of this invention are heteroaggregates, by which is meant that the aggregates comprise at least two polypeptides that have different sequences. Polypeptides capable of assembling to form heteroaggregates are referred to herein as “compatible”. In some embodiments a first polypeptide nucleates assembly of a second polypeptide, resulting in a heteroaggregate composed mainly of the second polypeptide. As described further below, polypeptides that assemble to form amyloids associated with biofilms are of particular interest.

The term “higher ordered” refers to an aggregate of at least 10 polypeptide subunits, or in some embodiments at least 15 polypeptide subunits, or in some embodiments at least 25 polypeptide subunits and is meant to exclude the many proteins that are known to include polypeptide dimers, tetramers, or other small numbers of polypeptide subunits in an active complex, although the peptides and polypeptides may form such complexes as well. The term “higher-ordered aggregate” also is meant to exclude random agglomerations of denatured proteins that can form in non-physiological conditions. Higher ordered aggregates of interest herein are commonly referred to in scientific literature by terms such as “amyloid”, “amyloid fibers”, “amyloid fibrils”, or simply as “fibers” or “fibrils”, and those terms are used interchangeably herein. The term “higher-ordered aggregate” is also used interchangeably herein with the noun “aggregate”. Polypeptides that assemble to form amyloid fibers are referred to herein as “amyloidogenic”. It will be understood than many polypeptides that can participate in formation of higher-ordered aggregates can exist in at least two conformational states, only one of which is typically found in the ordered aggregates or fibrils. The term “assembles” refers to the property of certain polypeptides to form ordered aggregates under appropriate conditions and is not intended to imply that the formation of higher ordered aggregates will occur under every concentration or every set of conditions. A peptide that, when present as part of a first polypeptide, can promote (e.g., accelerate or cause) assembly of a second polypeptide differing in sequence from the first polypeptide, so as to form fibers comprising both first and second polypeptides, is referred to herein as a “nucleating peptide” and its amino acid sequence will be referred to as a “nucleating sequence”. In some embodiments of the invention, a nucleating peptide is characterized in that its deletion (e.g., in part or in full) from a polypeptide significantly slows down or abolishes fiber assembly with a compatible polypeptide.

Amyloid fibers have a characteristic morphology under electron microscopy, are β-sheet rich, typically non-branching, and react characteristically with certain amyloid-specific dyes such as thioflavin T (ThT) and Congo red. Such dyes may be used to identify and/or detect amyloid fibers and thus serve as indicators of the formation or presence of such fibers in certain embodiments of the invention. In embodiments of interest herein, amyloid fibers are composed of two different polypeptide species, e.g., CsgA and CsgB. In some embodiments amyloid fibers are composed of more than two polypeptide species. The ratio of first polypeptide to second polypeptide in the fiber can vary. In some embodiments, the fiber is composed largely of the second amyloidogenic polypeptide. For example, in some embodiments the second polypeptide species constitutes at least 70%, at least 80%, at least 90%, or more of the fiber by weight, or, in some embodiments by number, of subunits. In other embodiments, the first polypeptide species constitutes at least 70%, at least 80%, at least 90%, or more of the fiber by weight, or, in some embodiments by number, of subunits. In one aspect, peptides that are derived from a first amyloidogenic polypeptide, and to which a second amyloidogenic polypeptide having a different sequence to the first amyloidogenic polypeptide binds to form a higher ordered aggregate are provided. In some embodiments the first and second polypeptides are at least 50%, 60%, 70%, 80%, 90%, or up to 95% identical. In some embodiments the first and second amyloidogenic polypeptides are no more than 50% identical, e.g., between 20% and 40% identical. In some embodiments, the presence of the first polypeptide or an aggregation domain derived from the first polypeptide greatly accelerates or is required for formation of an amyloid comprising the second polypeptide. Either or both of the polypeptides may contain multiple aggregation domains, which can be identical or different in sequence.

Provided herein is a collection that comprises a plurality of peptides, wherein the peptides are portions of a first amyloidogenic polypeptide that is prone to form aggregates with a second amyloidogenic polypeptide of different sequence under appropriate conditions. In some embodiments the first amyloidogenic polypeptide is any polypeptide that can form heteroaggregates comprised in part of a second amyloidogenic polypeptide. In some embodiments of interest the first and second amyloidogenic polypeptides are at least 70%, 80%, 85%, 90%, 95%, 96%, 97%, 98%, 99%, or 100% identical to polypeptides that assemble to form amyloids present in biofilms. In some embodiments of particular interest the first amyloidogenic polypeptide is a CsgB polypeptide and the second amyloidogenic polypeptide is a CsgA polypeptide. In some embodiments the first amyloidogenic polypeptide is any naturally occurring polypeptide wherein heteroaggregates formed in part from the polypeptide and/or in part from fragments of the polypeptide play a role in disease, e.g., in mammals such as humans, non-human primates, domesticated animals, rodents such as mice or rats, etc. In some embodiments the first polypeptide is at least 70%, 80%, 85%, 90%, 95%, 96%, 97%, 98%, 99%, or 100% identical to such a naturally occurring polypeptide.

The collection may contain, e.g., up to 10, 50, 100, 150, 200, 250, or more different peptides. The sequences of the peptides may collectively encompass between 20-100% of the complete polypeptide sequence, e.g., 30-100%, 40-100%, 50-100%, 60-100%, 70-100%, 80-100%, or 90-100% of the full length sequence. The peptides may be, e.g., 6-12, 8-15, 10-20, 10-30, 20-30, 30-40, or 40-50 amino acids in length. In some embodiments, the peptides overlap in sequence by between, e.g., 1-25 residues, e.g., between 5-20 residues, or between 10-15 residues. In some embodiments, the peptides “scan” at least a portion of the polypeptide, i.e., the starting positions of the peptides with respect to the polypeptide are displaced from one another (“staggered”) by X residues where X is, for example, between 1-10 residues or between 1-6 residues or between 1-3 residues. In one embodiment, the starting positions of the peptides with respect to the polypeptide sequence are staggered by 1 amino acid. For example, a first peptide corresponds to amino acids 1-20; a second peptide corresponds to amino acids 2-21; a third peptide corresponds to amino acids 3-22, etc. In another embodiment, the starting positions of the peptides with respect to the polypeptide sequence are staggered by 2 amino acids. For example, a first peptide corresponds to amino acids 1-20; a second peptide corresponds to amino acids 3-22; a third peptide corresponds to amino acids 5-23, etc. The collection need not include a peptide that comprises the N-terminal or C-terminal amino acid(s) of the polypeptide. For example, a signal sequence could be omitted. The collection could span any N-terminal, C-terminal, or internal portion of the polypeptide. In some embodiments the peptides have a detectable label, a reactive moiety, a tag, a spacer, or a crosslinker linked thereto. The peptides need not all be the same length and need not all fall within any single range of lengths. The peptides can be provided in individual receptacles, wells, locations, or in any manner in which peptides having distinct sequences are separated or distinguishable from each other. In some embodiments the peptides are provided in individual wells of a microwell plate (e.g., a 96, 384, or 1536 well plate). It will be appreciated that a receptacle, well, location, etc., will typically contain multiple molecules of a given peptide. Not all such molecules need be identical. For example, a peptide preparation in a given receptacle, well, or location may consist of at least 70%, 80%, 90%, 95%, 98%, 99%, or more peptides having an identical sequence. It will be appreciated that during synthesis errors and truncated peptides can occur, resulting in preparations having less than 100% uniformity of sequence.

Further provided herein is an array that comprises a collection of peptides as described above, wherein the array comprises a surface having a plurality of discrete regions (“features”), each of which comprises a peptide. It will he understood that each feature comprises multiple peptide molecules having the same sequence. In some embodiments a feature comprises two or more distinct peptides. The surface could be made of any suitable solid or semi-solid material known in the art, e.g., glass, plastic (e.g., polystyrene, polycarbonate), metal, silicon, semi-solid polymers, etc. The array may include up to 10, 100, 1000, or more features. The features may be disposed in close proximity to one another on a surface such as a slide, wherein they are not separated into individual wells, or on a membrane or filter. In some embodiments the array is microfabricated. Methods for making such arrays are known in the art and include a wide variety of printing techniques (e.g., contact or non-contact printing), automated or manual mechanical deposition, as well as synthesis in situ. See, e.g., U.S. Pat. Nos. 6,630,358; 6,475,809; 6,815,078; 7,067,322. In some embodiments the array is a microengraved array and may fit on a glass slide (1 inch×3 inch). In some embodiments an array of microwells is fabricated by photolithography, e.g., soft lithography of slabs of poly(dimethylsiloxane) or another suitable polymer. The peptides may be covalently or noncovalently attached to the surface. They may be directly attached to the surface or attached via a linker. In some embodiments the surface is modified to contain a binding moiety or reactive moiety that binds to or reacts with the peptide. The surface density and number of peptide molecules in each feature, the feature size, and the distance between features, etc. could vary. For example, the peptides can be deposited in a solution whose concentration is between 1 μm and 5 μm, e.g., about 2.5 μm. The peptide density may be, e.g., about 15-150 fmol/mm². In some embodiments the peptide is deposited in a solution whose concentration ranges between 0.001 to 1000 times either of the afore-mentioned ranges, e.g., between 0.01 to 100 times either of the afore-mentioned ranges, e.g., between 0.1 to 10 times or between 0.5 to 5 times either of the afore-mentioned ranges.

In some embodiments, the peptides are attached to particles which in some embodiments are distinguishable from one another. The particles may be coded by any of a variety of methods. For example, they may incorporate different detectable moieties such as fluorescent dyes; they may include different oligonucleotide or peptide tags that allow their differential detection and/or isolation, etc. In other embodiments, the peptides can be provided in any assay format that allows for multiplexed protein detection and/or measurement. Peptides may be covalently attached to thiolated PEG, surface coated polystyrene/ silica beads, colloidal gold, glass, plastic, hydrogels, etc., and presented in various formats (multiwell well plates, Eppendorf tubes, etc). Once a peptide of interest is identified, the peptide can be used, e.g., to screen for agents that inhibit aggregate formation, without the other members of the array. It may be desirable to use negative controls, e.g., peptides from the array that did not show appreciable ability to seed aggregation.

The invention further provides a composition comprising an array as described herein and a second amyloidogenic polypeptide, wherein the second amyloidogenic polypeptide is in contact with the array. As used herein, a polypeptide is considered to be “in contact with an array” if the polypeptide is present in a liquid medium, e.g., an aqueous medium, that is in contact with the array surface to which the peptides are attached. In some embodiments the second polypeptide is at least partly in solution in the liquid medium. In some embodiments the concentration of peptide in a solution deposited to form the arrayed features is greater than the concentration of the polypeptide in solution. In some embodiments the concentration of peptide in a deposited solution is less than the concentration of the polypeptide in solution. In some embodiments the concentration of peptide in a deposited solution is between 1 and 10,000 times the concentration of the polypeptide in solution, e.g., between 10 and 5,000 the concentration of the polypeptide in solution, between 100 and 1000 times the concentration of the polypeptide in solution, etc.

The invention provides methods of identifying a peptide that seeds assembly of an amyloidogenic polypeptide. One such method comprises: providing an array including a plurality of peptides, wherein the peptides are fragments of a first polypeptide that forms aggregates that comprise the first polypeptide and a second polypeptide; contacting the array with the second polypeptide; and identifying a peptide that nucleates assembly of the second polypeptide to form a higher ordered aggregate, thereby identifying a peptide that seeds assembly of the second polypeptide. The contacting can take place under a variety of conditions of temperature, pH, osmolarity, salt concentration, etc. In some embodiments the conditions resemble physiological conditions, e.g., conditions under which the first and second polypeptides assemble in nature. The Examples provide suitable conditions, but one of skill in the art will appreciate that the conditions could be varied. A suitable pH may be 5-10, e.g., 6-9, e.g., about 7-7.5. A suitable salt concentration may be, e.g., 100 mM to 200 mM, e.g., 140-160 mM. A suitable temperature may be 20-50° C., e.g., 30-45° C., e.g., 35-40° C., or 37° C. The second polypeptide is provided in soluble form. The second polypeptide may be present in solution as monomers, dimers, or oligomers, e.g., including 3-5 individual molecules. In some embodiments the solution includes a mixture of monomers, dimers, and oligomers. In some embodiments at least 25%, 50%, 75%, or 90% of the polypeptide by weight is present in monomeric form. In some embodiments the second polypeptide is denatured prior to contacting with the peptides. The contacting could take place over a time period ranging from 10 minutes to several hours, days, or longer, e.g,, between 1 and 24 hours, between 2 and 12 hours, between 24 and 48 hours, etc. In some embodiments cells that secrete the second polypeptide are provided in the composition.

In certain embodiments of particular interest the invention relates to polypeptides that promote formation of biofilms. In some embodiments of interest the first and second amyloidogenic polypeptides are at least 70%, 80%, 85%, 90%, or 95% identical to polypeptides that assemble to form amyloids present in biofilms e.g., bacterial polypeptides that assemble to form amyloid fibers such as curli. Curli are the major proteinaceous component of a complex extracellular matrix produced by many bacteria, e.g., many Enterobacteriaceae such as E. coli and Salmonella spp. (Barnhart M M, Chapman M R. Annu Rev Microbiol., 60:131-47, 2006). Other biofilm-forming bacteria of interest include Klebsiella, Pseudomonas, Enterobacter, Serratia, Citrobacter, Proteus, Yersinia, Citrobacter, Shewanella, Agrobacter, Campylobacter, etc. Curli fibers are involved in adhesion to surfaces, cell aggregation, and biofilm formation. Curli also mediate host cell adhesion and invasion, and they are potent inducers of the host inflammatory response. Curli exhibit structural and biochemical properties of amyloids, e.g., they are nonbranching, β-sheet rich fibers that are resistant to protease digestion and denaturation by 1% SDS and bind to amyloid-specific moieties such as thioflavin T, which fluoresces when bound to amyloid, and Congo red, which produces a unique spectral pattern (“red shift”) in the presence of amyloid. Polypeptides that assemble to form curli are of interest at least in part because of their association with animal and human disease. Bacterial polypeptides that promote formation of biofilms present in a variety of natural habitats are also of interest. For example, in a recent study bacteria producing extracellular amyloid adhesins were identified within several phyla: Proteobacteria (Alpha-, Beta-, Gamma- and Deltaproteobacteria), Bacteriodetes, Chloroflexi and Actinobacteria (Larsen, P., et al., Environ Microbial., 9(12):3077-90, 2007). Particularly in drinking water biofilms, a high number of amyloid-positive bacteria were identified. Bacteria of interest may be gram-negative or gram-positive. In some embodiment bacteria of interest are rods. In some embodiments they are aerobic. In some embodiments they are facultative anaerobes or anaerobes.

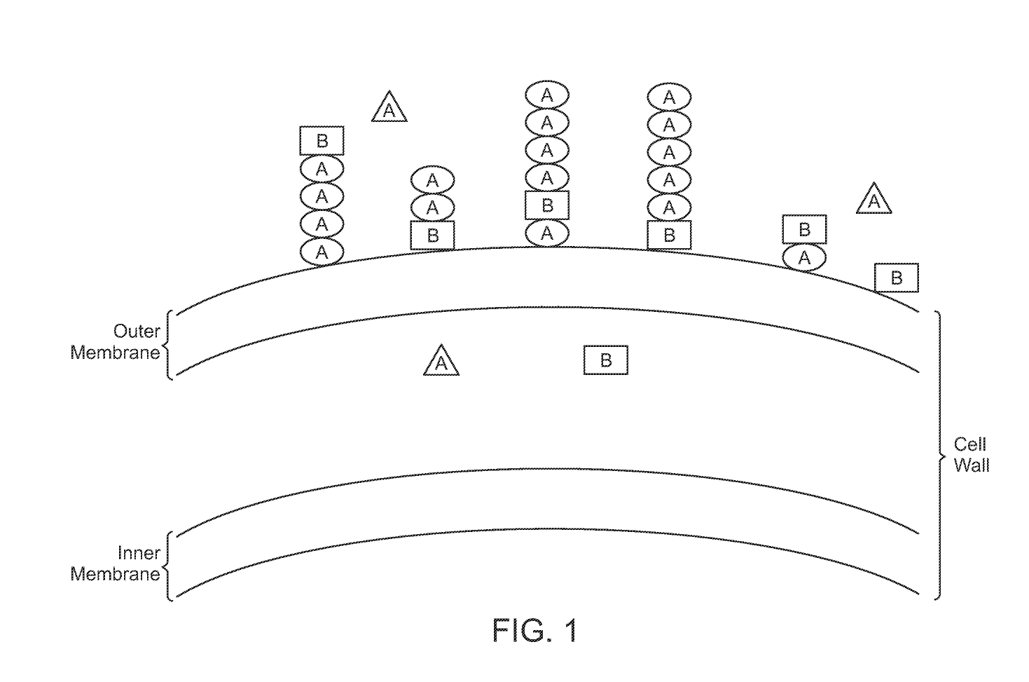

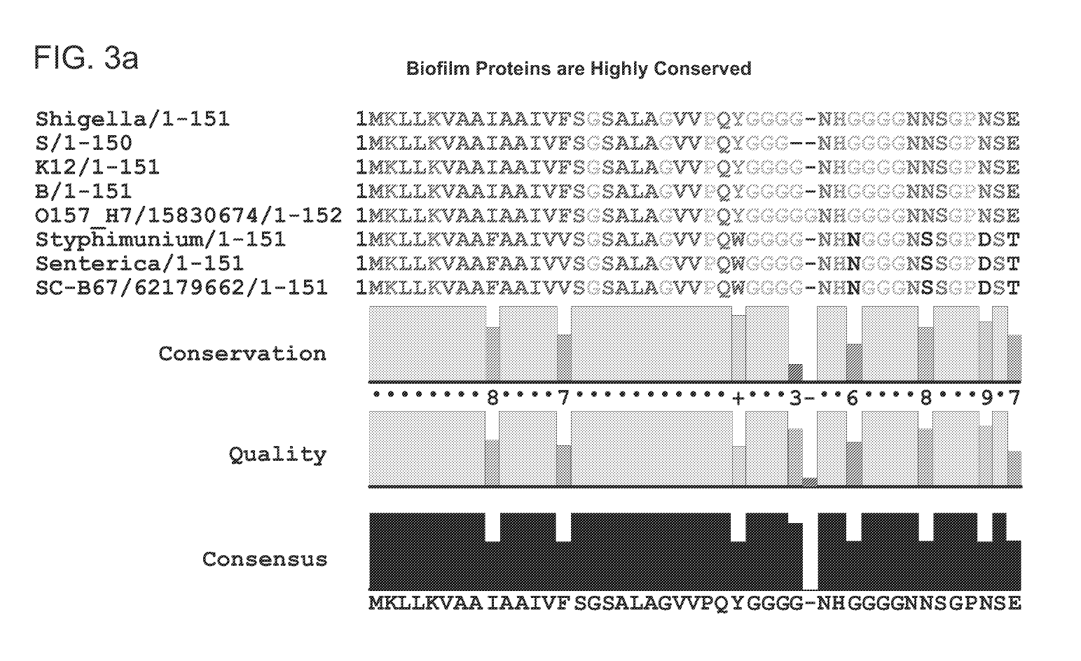

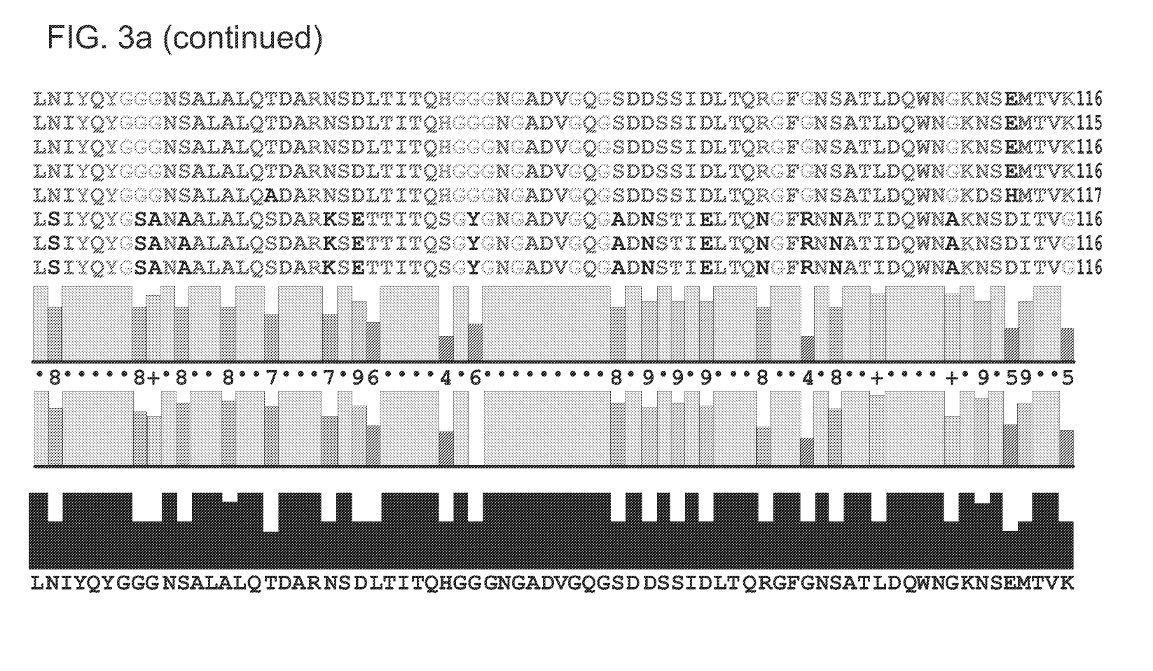

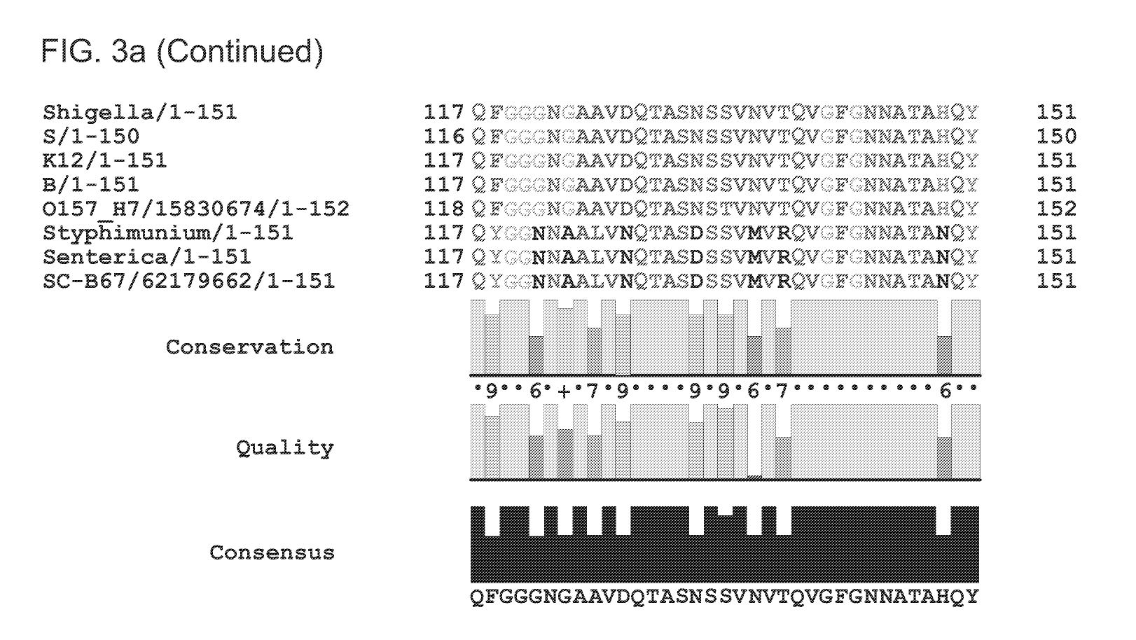

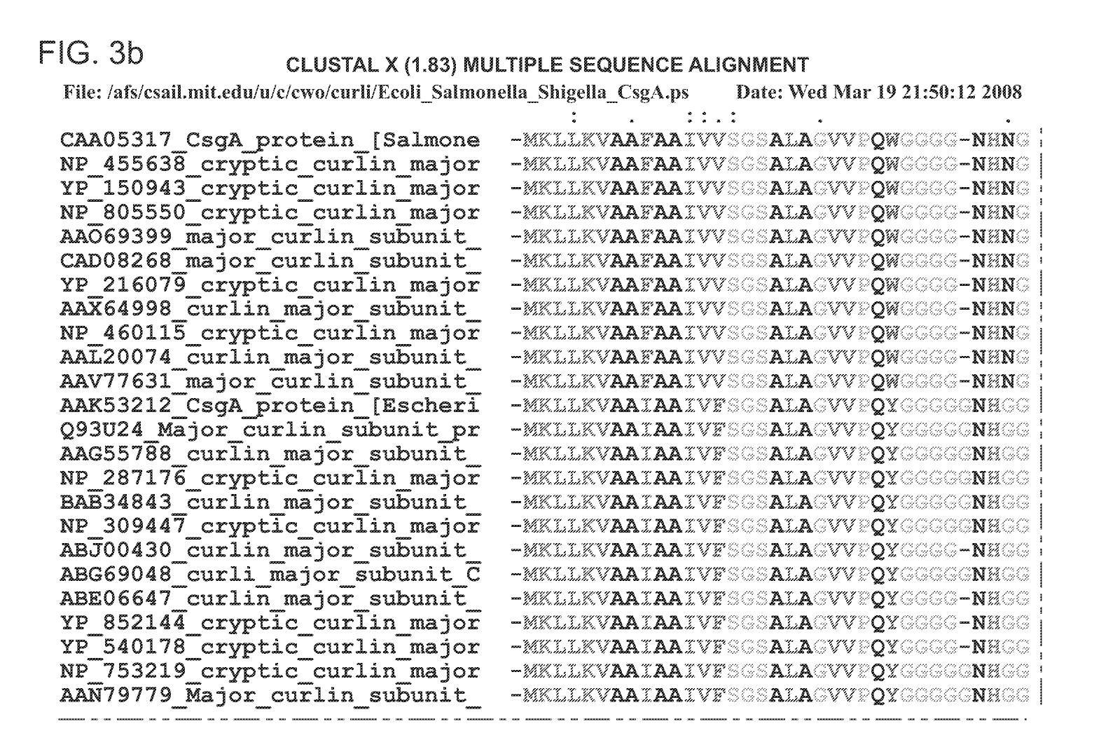

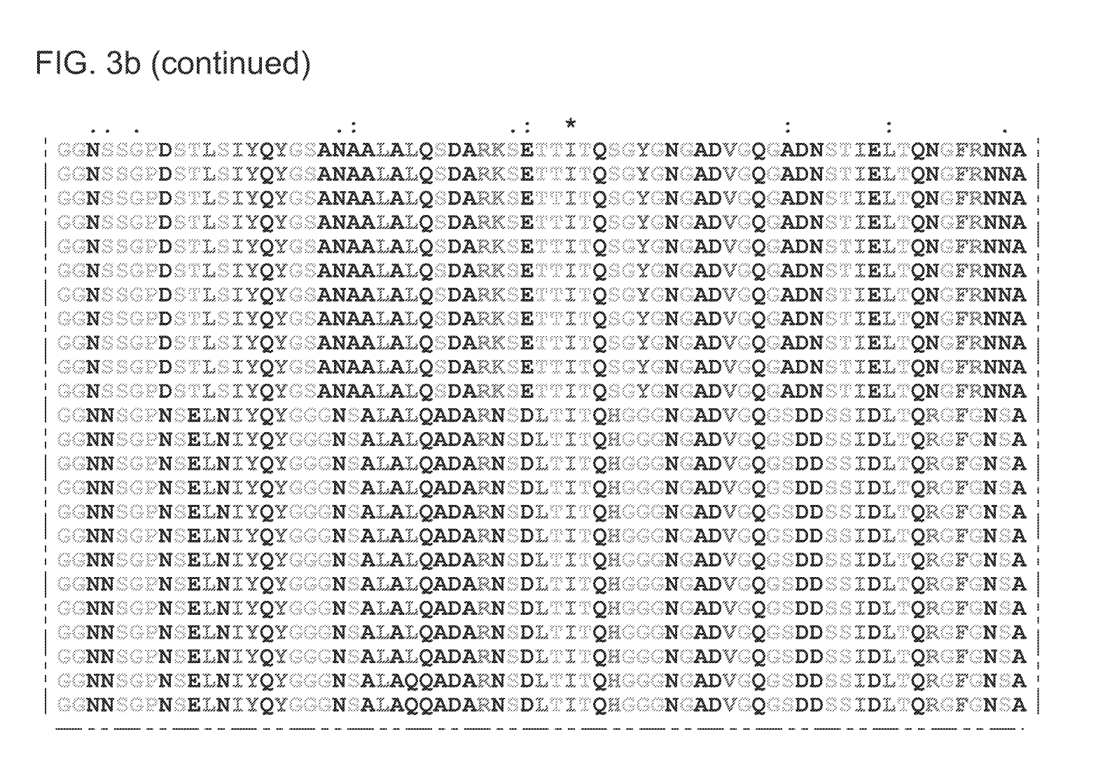

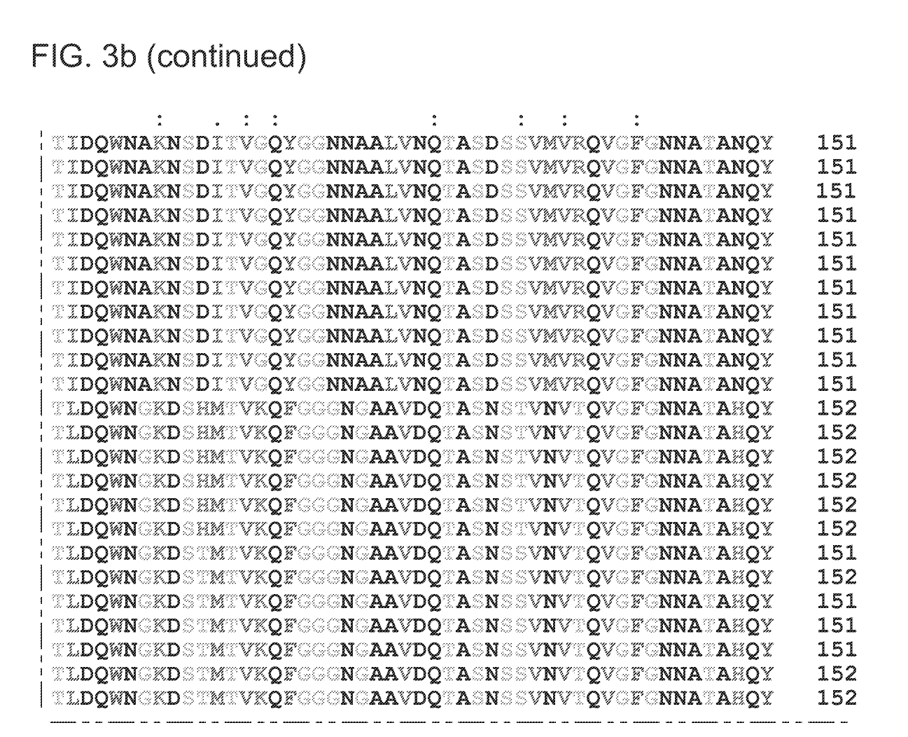

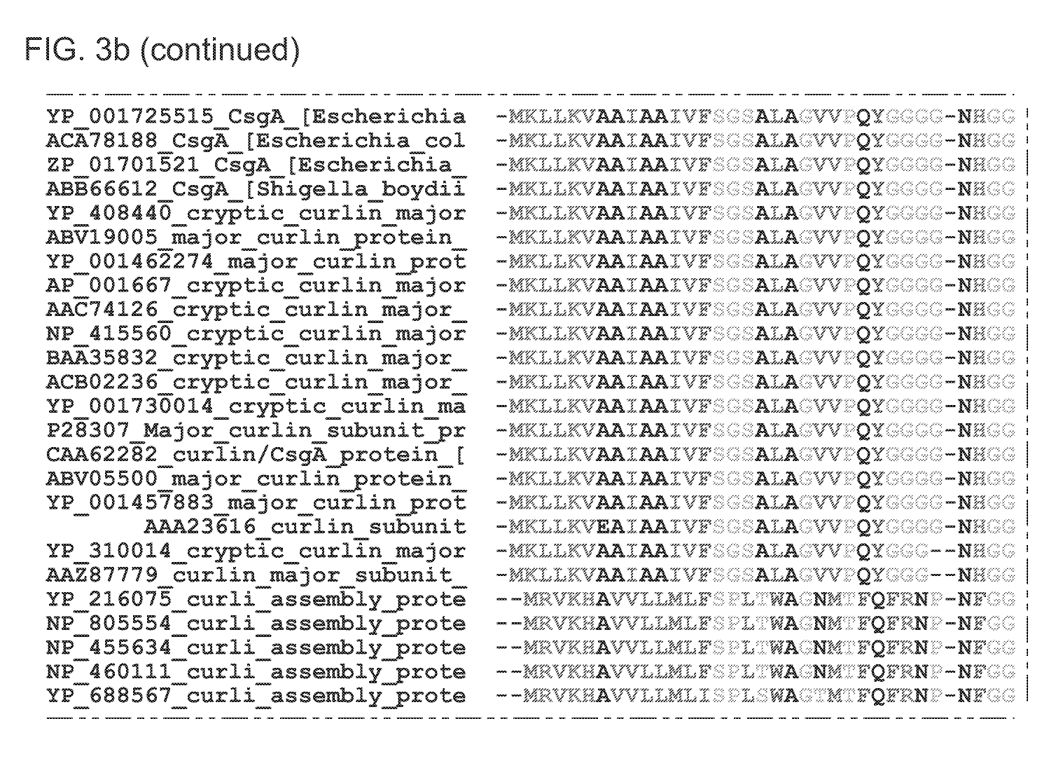

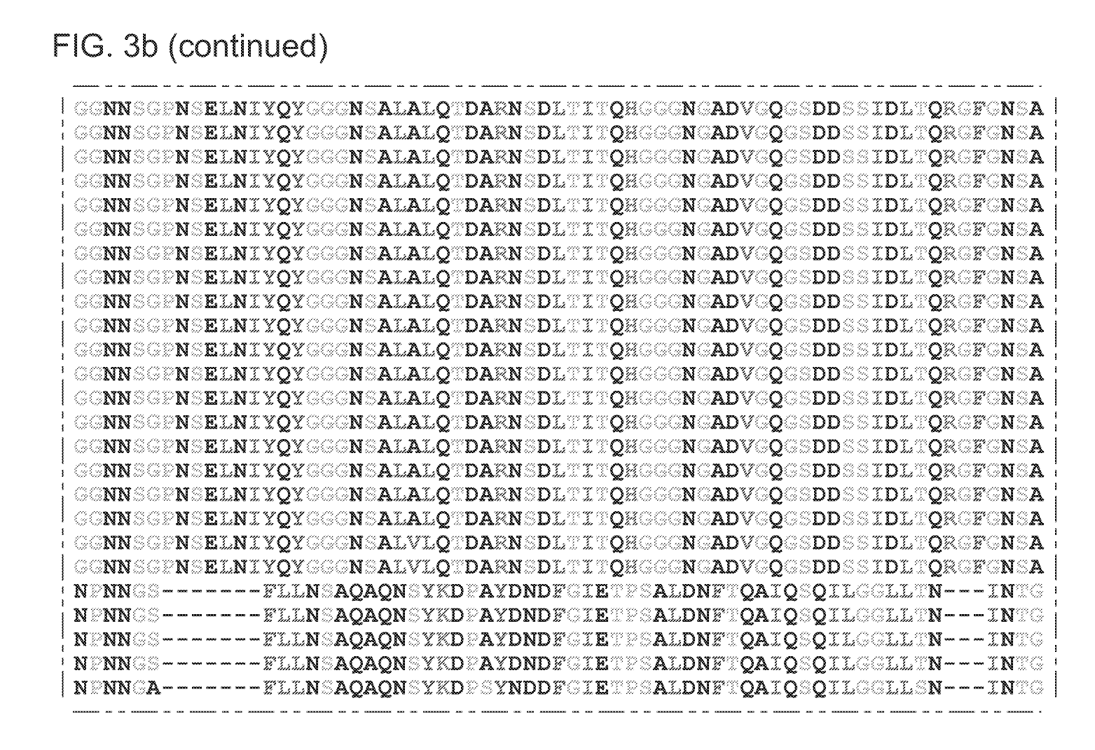

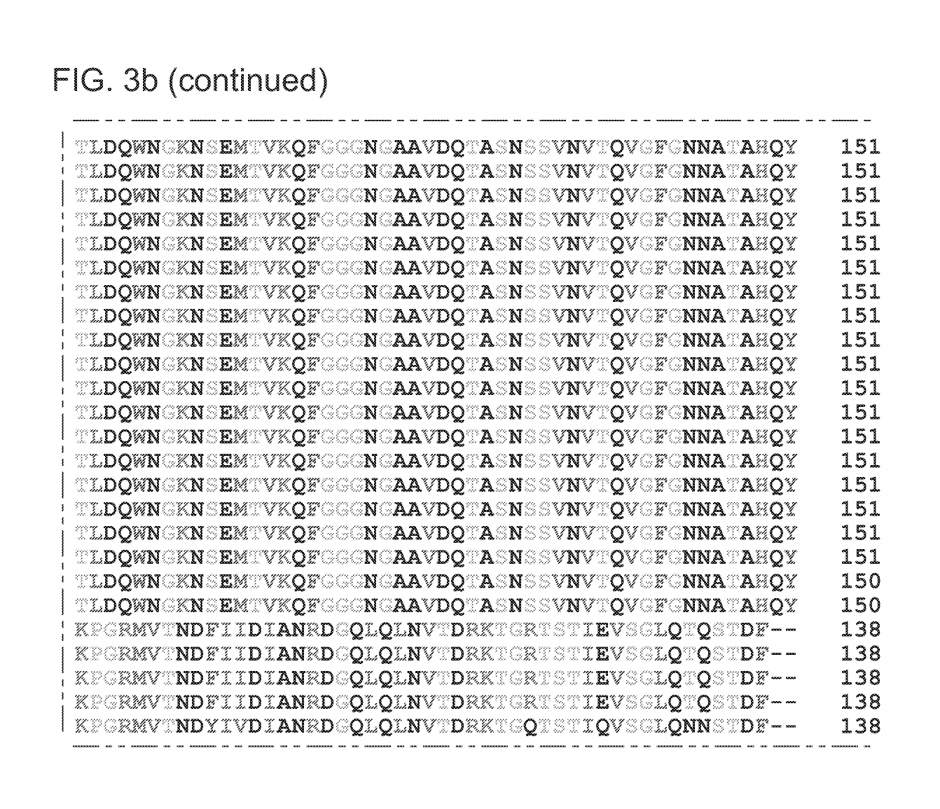

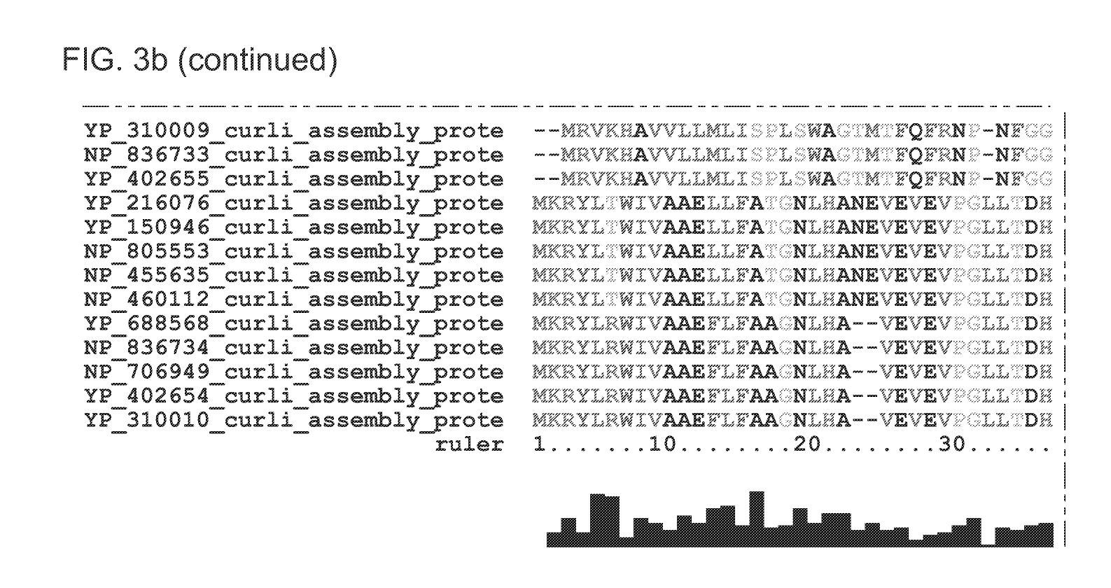

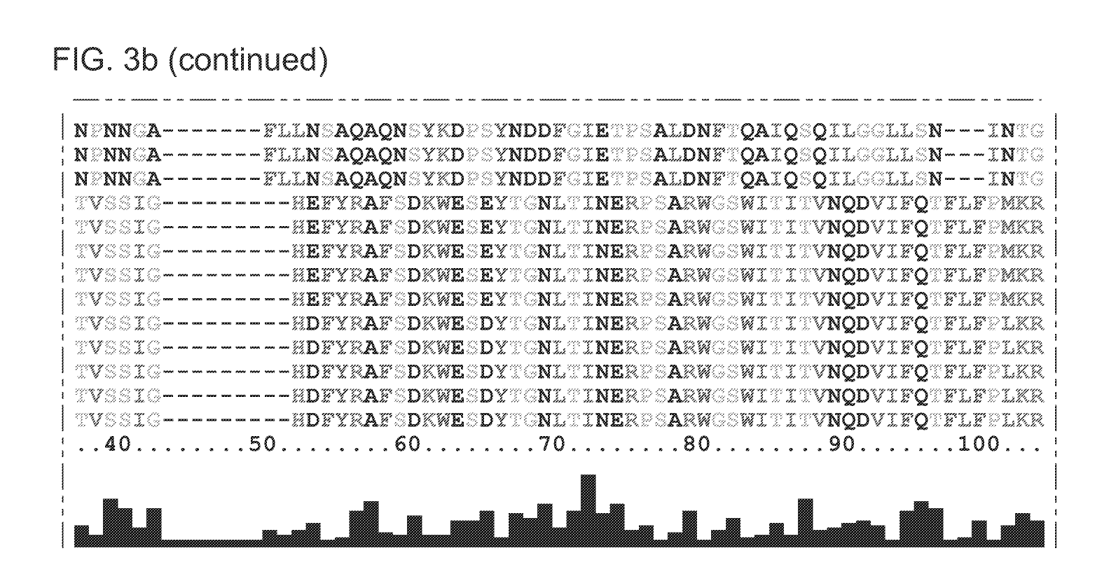

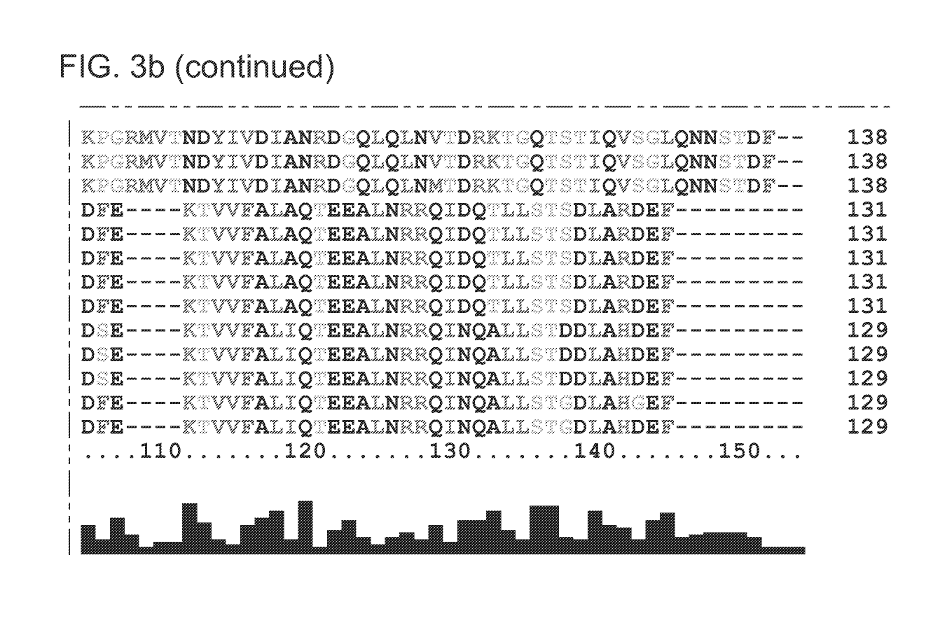

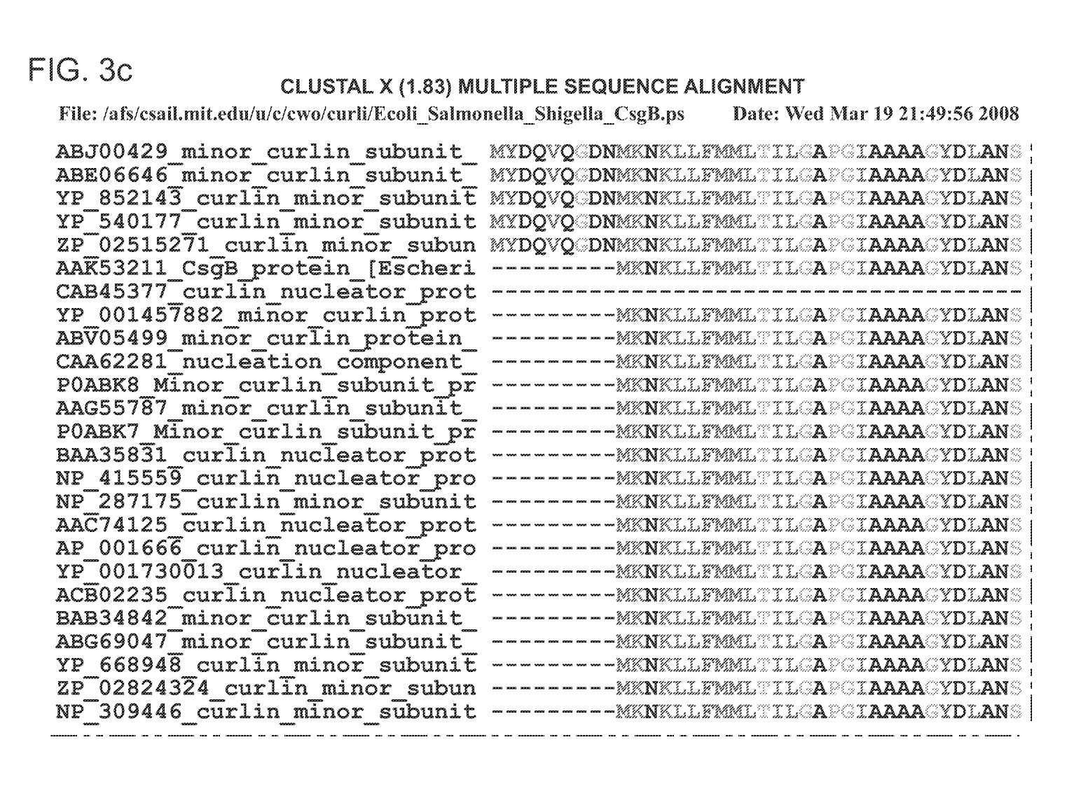

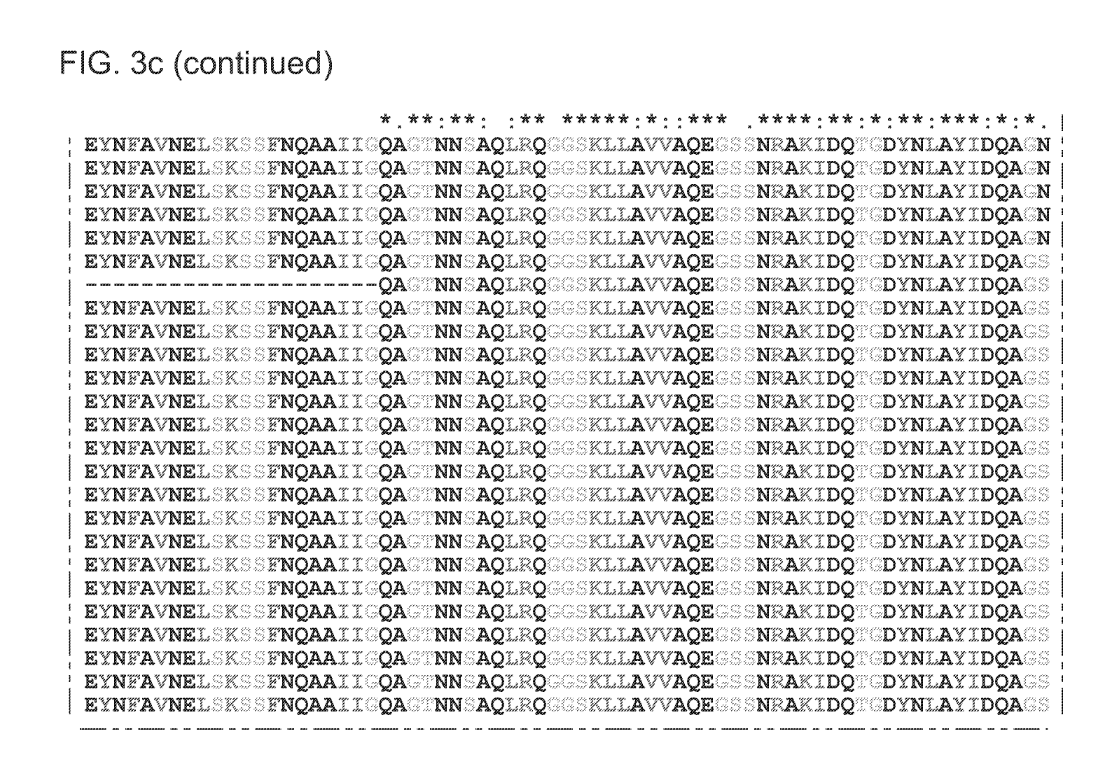

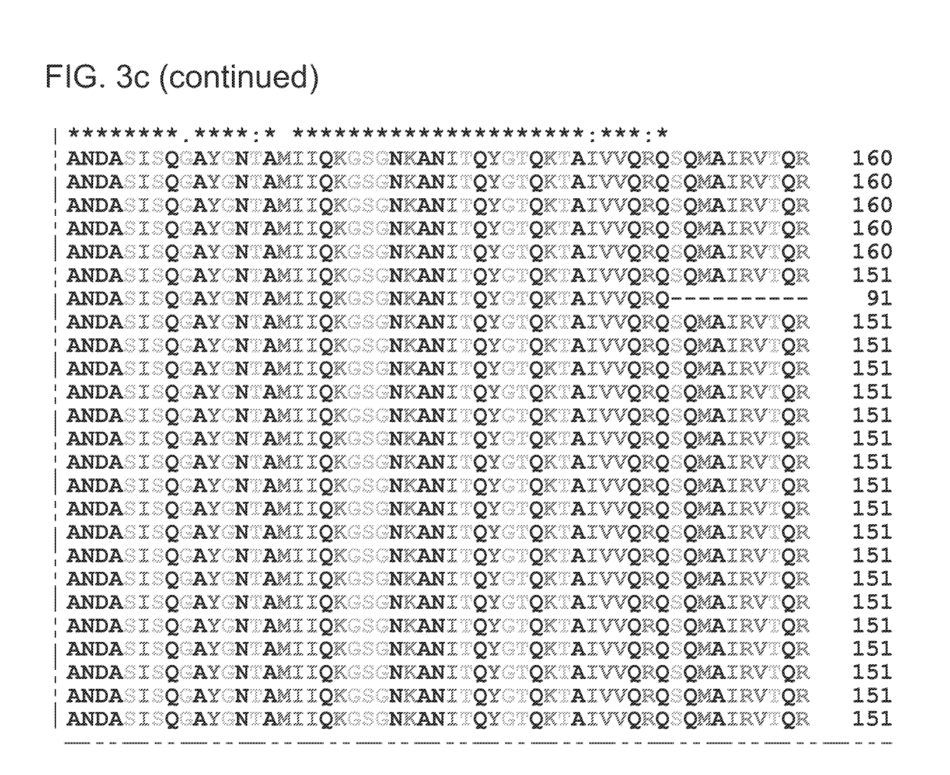

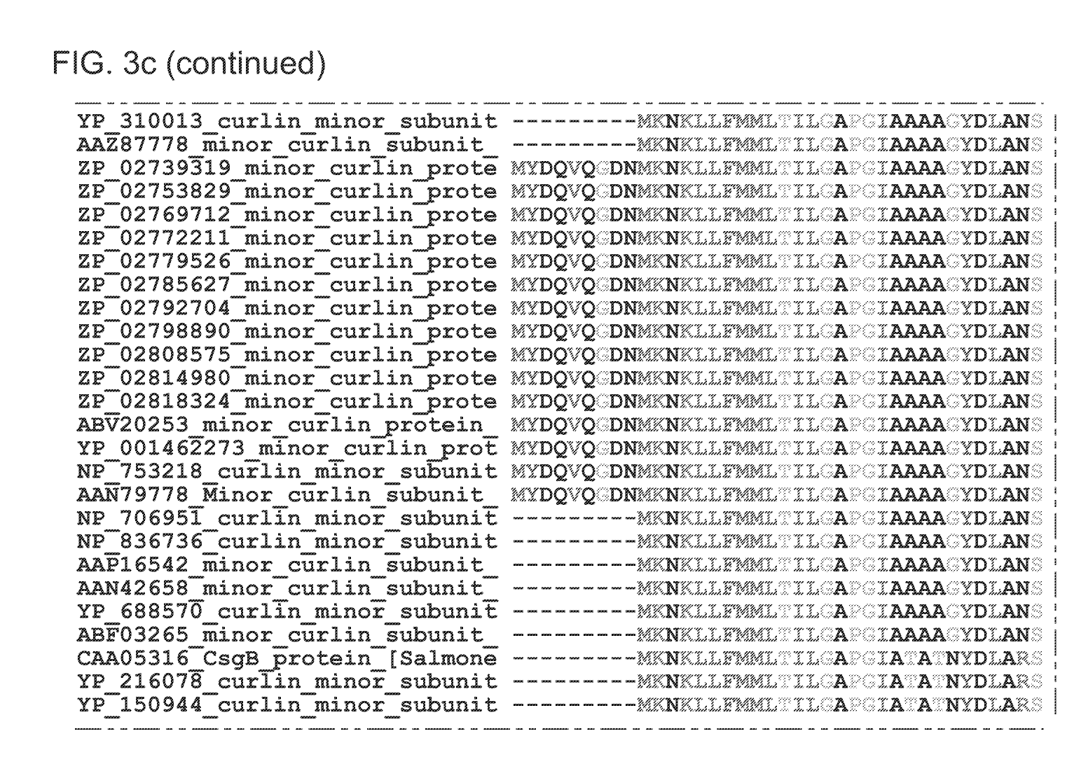

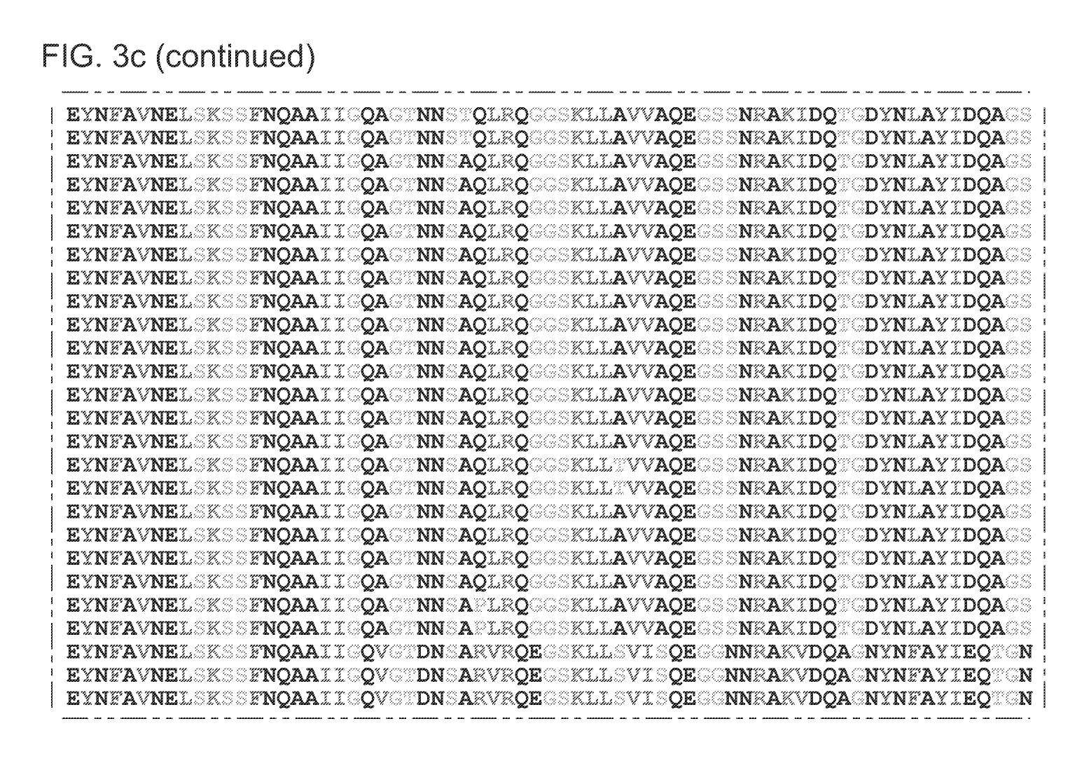

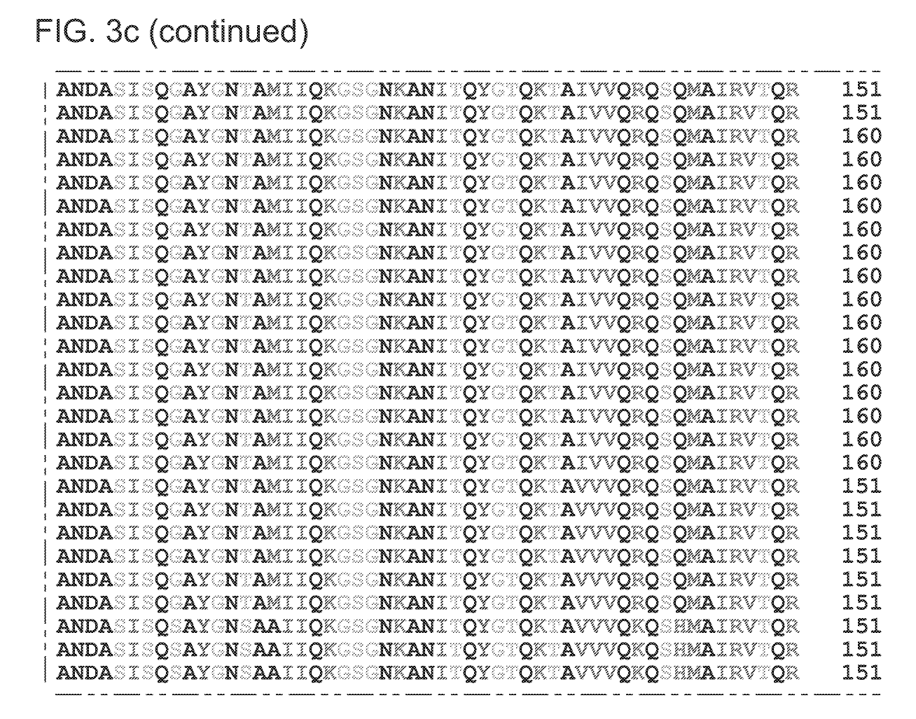

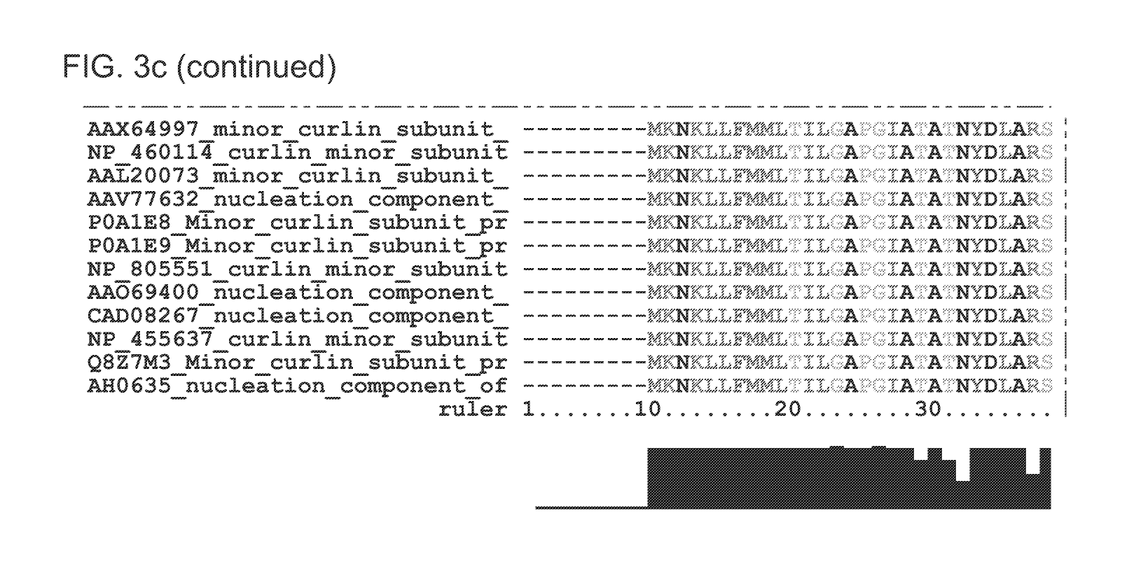

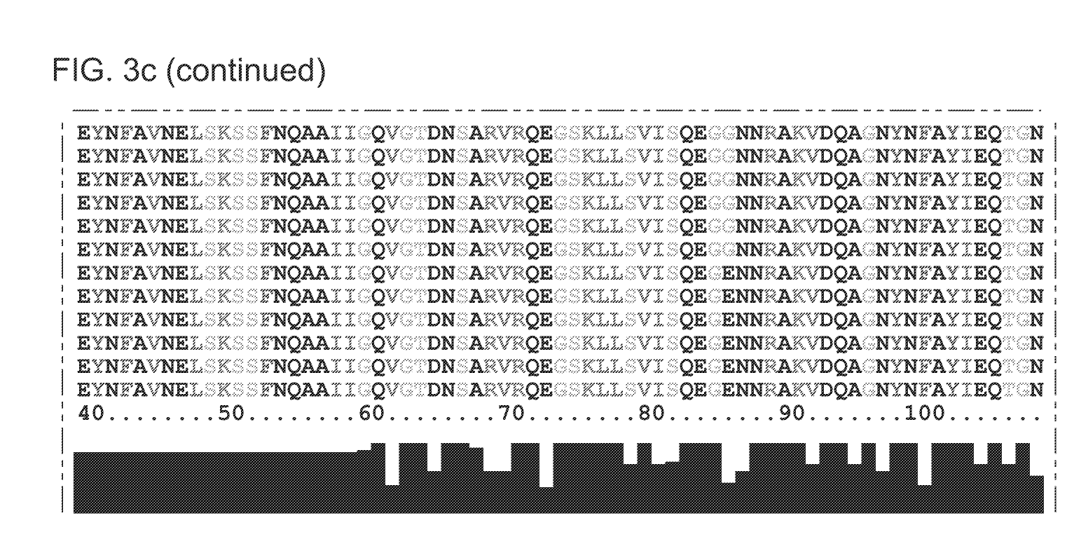

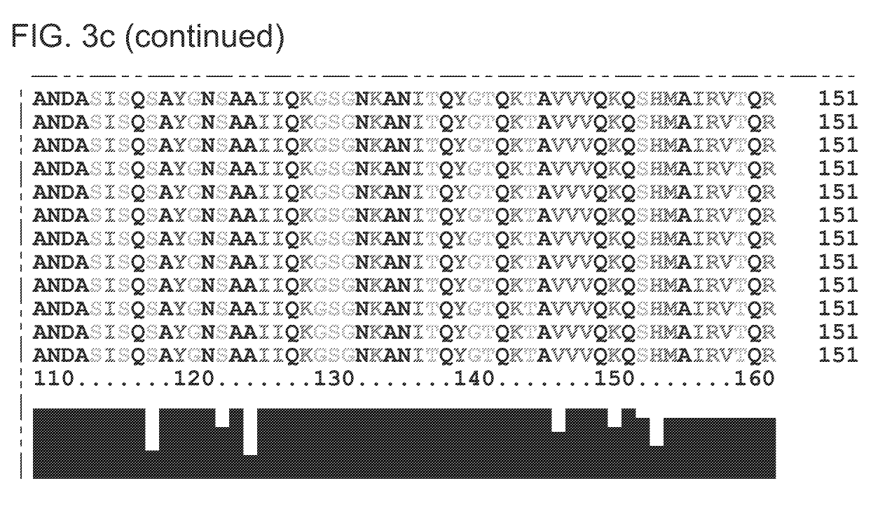

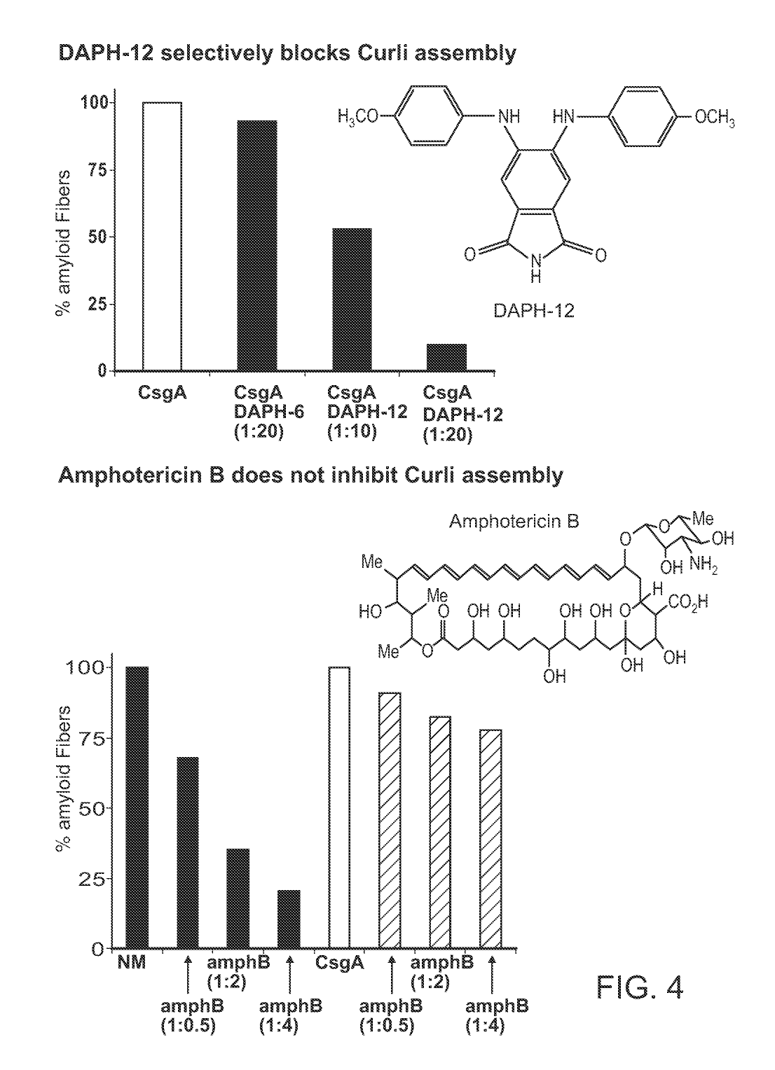

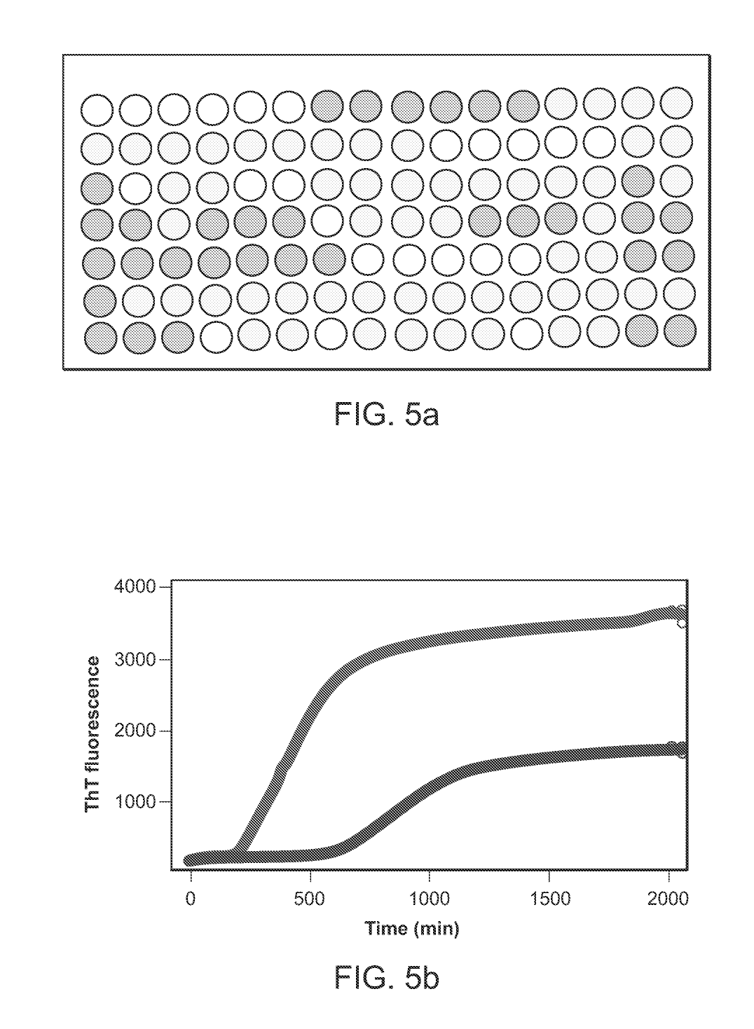

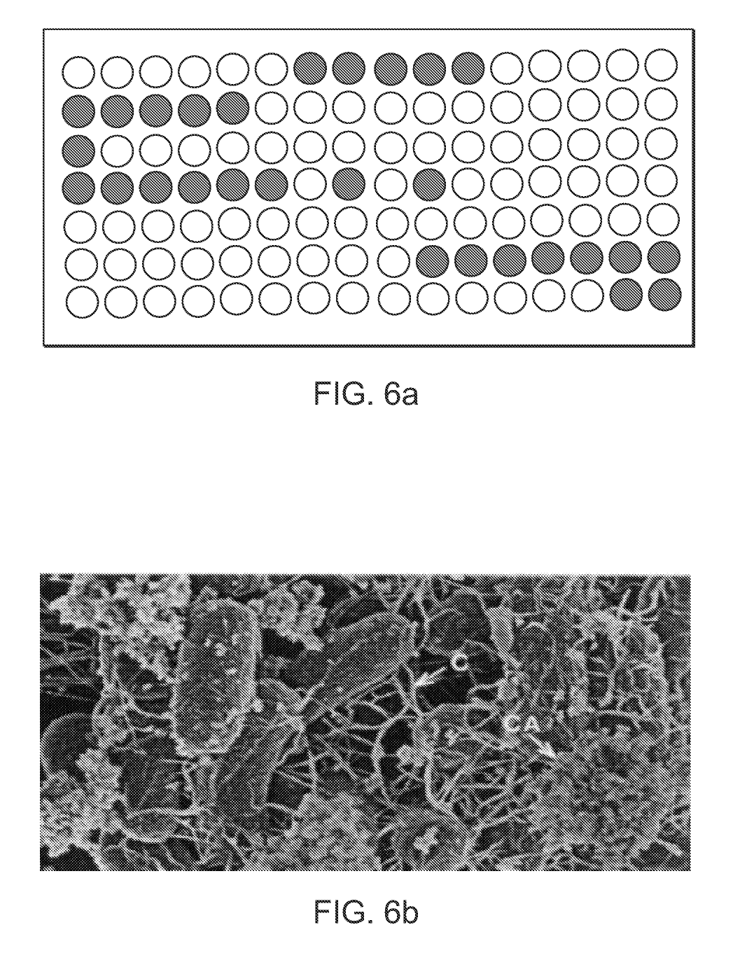

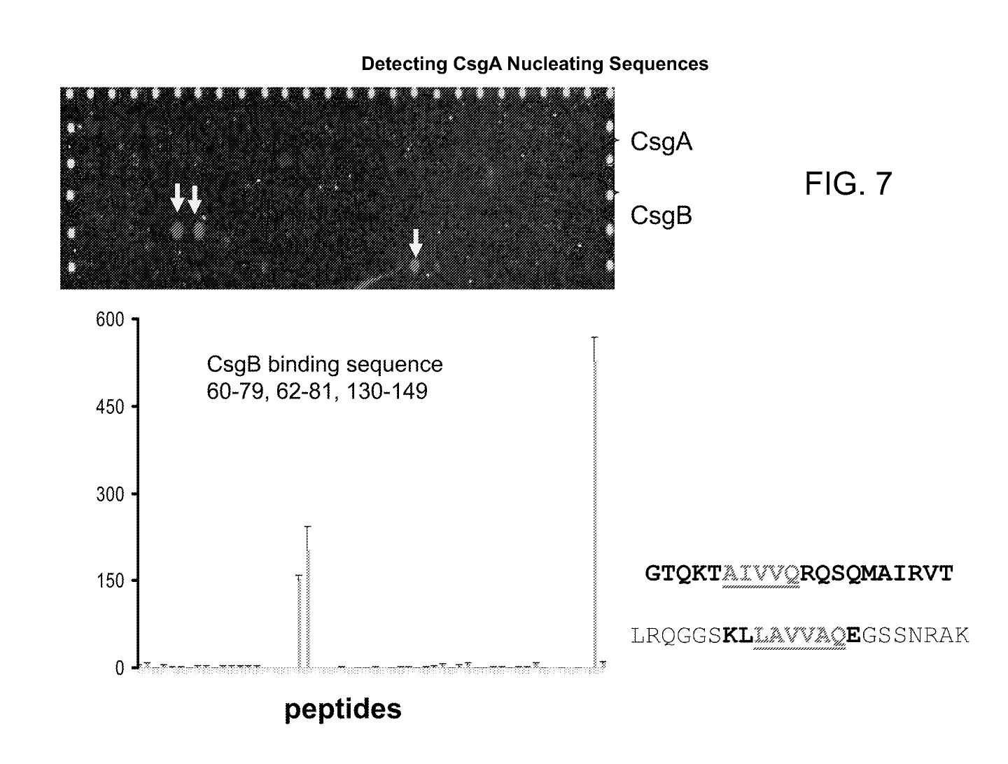

In nature, curli are assembled by a process in which the major curlin subunit polypeptide, CsgA, is nucleated into a fiber by the minor curlin subunit polypeptide, CsgB (see FIG. 1 for schematic diagram). CsaA and CsgB are about 30% identical at the amino acid level and contain five-fold internal symmetry characterized by conserved polar residues, The assembly process is believed to involve addition of soluble polypeptides to the growing fiber tip. Thus both subunits are incorporated into the fiber, although CsgA is the major protein constituent. In living bacteria, curli formation likely involves activities of several additional polypeptides encoded by other Csg genes (CsgD, CsgE, CsgF, CsgG), but these polypeptides are not required for curli formation in vitro. Sequences of CsgA and CsgB from a large number of bacteria have been identified. Exemplary CsgA and CsgB sequences are shown in FIGS. 3, 7, and 8. One of skill in the art will readily be able to find CsgA and CsgB sequences by searching databases such as GenBank publicly available through the National Center for Biotechnology Information (NCBI). See ncbi.nlm.nih.gov.

The present invention is based in part on the discovery that small sequence elements that initiate curli fiber formation can be identified within the sequences of bacterial CsgB polypeptides using peptide arrays. Further, it was found that these sequence elements mimic the in vivo assembly of curli fibers in that, while peptides whose sequence is found within the sequence of CsgB efficiently nucleated assembly of CsgA into amyloid, peptides whose sequence is found within the sequence of CsgA did not detectably do so under the conditions employed. As described in the Examples, specific peptides within E. coli CsgB nucleated assembly of amyloid fibers when arrays having the peptides attached thereto were incubated in the presence of CsgA. Results thus demonstrate that short peptide portions of bacterial biofilm forming proteins, lacking the context provided by some or all of the remainder of the full length polypeptide from which they were derived, bind directly to full length polypeptides and promote their assembly to form higher order aggregates, e.g., fibrils. Furthermore, these results show binding of the polypeptide to the peptide and aggregate formation can take place when the peptide is attached to a support. Notably, the results demonstrate that peptide arrays can be used to identify peptide portions of a first polypeptide that nucleate assembly of a second polypeptide with a distinct sequence. These peptides, compositions comprising the peptides, and uses thereof are aspects of the invention.

“CsgA polypeptide” as used herein encompasses any polypeptide whose sequence comprises or consists of the sequence of a naturally occurring bacterial CsgA polypeptide. The term also encompasses polypeptides that are variants or fragments of a polypeptide whose sequence comprises or consists of the sequence of a naturally occurring bacterial CsgA polypeptide, which are referred to as “CsgA polypeptide variants” and “CsgA polypeptide fragments”, respectively In some embodiments a CsgA polypeptide variant is at least 50%, 60%, 70%, 80%, 85%, 90%, 95%, 96%, 97%, 98%, 99%, or 100% identical to or similar to a naturally occurring CsgA polypeptide across the length of the CsgA polypeptide variant. In some embodiments the CsgA polypeptide fragment or variant is at least 50%, 60%, 70%, 80%, 90%, 95%, 98%, or 100% as long as a naturally occurring CsgA polypeptide. In some embodiments a fragment is at least 8-10 amino acids long. In some embodiments a CsgA polypeptide is wild type at one, more, or all of the following positions: 49, 54, 139, 144 (where amino acid numbering is based on the E. coli CsgA sequence). In some embodiments the CsgA polypeptide has a substitution at one or more of the foregoing positions. “CsgB polypeptide” as used herein encompasses any polypeptide whose sequence comprises or consists of the sequence of a naturally occurring bacterial CsgB polypeptide. The term also encompasses polypeptides that are variants or fragments of a polypeptide whose sequence comprises or consists of the sequence of a naturally occurring bacterial CsgB polypeptide. Such variants and fragments are referred to as “CsgB polypeptide variants” and “CsgB polypeptide fragments”, respectively. In some embodiments a CsgB polypeptide variant is at least 50%, 60%, 70%, 80%, 85%, 90%, 95%, 96%, 97%, 98%, 99%, or 100% identical to or similar to a naturally occurring polypeptide across the length of the CsgB polypeptide variant. In some embodiments the CsgB polypeptide fragment or variant is at least 50%, 60%, 70%, 80%, 90%, 95%, 98%, or 100% as long as a naturally occurring CsgB polypeptide. In some embodiments the CsgA or CsgB polypeptide variant lacks about 10-20 amino acids from the N-terminus, C-terminus, or both, as compared with a naturally occurring CsgA or CsgB polypeptide. The invention provides embodiments that relate specifically to polypeptides whose sequence comprises or consists of the sequence of a naturally occurring bacterial CsgA polypeptide. The invention provides embodiments that relate specifically to polypeptides whose sequence comprises or consists of the sequence of a naturally occurring bacterial CsgB polypeptide. The invention also provides embodiments that relate to any subset of, or range within, the variants or fragments defined above. For example, the invention provides embodiments that relate to CsgA polypeptides that are at least 50% as long as a naturally occurring CsgA polypeptide and at least 90% identical to the naturally occurring CsgA polypeptide across their length and embodiments that relate to CsgB polypeptides that are at least 50% as long as a naturally occurring CsgB polypeptide and at least 90% identical to the naturally occurring CsgB polypeptide across their length.

Any of the peptides, polypeptides, nucleic acids, aggregates, etc., disclosed herein may be “isolated” or “purified”. “Isolated” is used herein to indicate that the material referred to is (i) separated from one or more substances with which it exists in nature (e.g., is separated from at least some cellular material, separated from other polypeptides, separated from its natural sequence context), and/or (ii) is produced by a process that involves the hand of man such as recombinant DNA technology, chemical synthesis, etc.; and/or (iii) has a sequence, structure, or chemical composition not found in nature. “Purified” as used herein denote that the indicated nucleic acid or polypeptide is present in the substantial absence of other biological macromolecules, e.g., polynucleotides, proteins, and the like. In one embodiment, the polynucleotide or polypeptide is purified such that it constitutes at least 90% by weight, e.g., at least 95% by weight, e.g., at least 99% by weight, of the polynucleotide(s) or polypeptide(s) present (but water, buffers, ions, and other small molecules, especially molecules having a molecular weight of less than 1000 daltons, can be present).

The terms “polypeptide” and “protein” are be used interchangeably herein. A peptide is a relatively short polypeptide, typically between 2 and 60 amino acids in length, e.g., between 5 and 50 amino acids in length. Polypeptides and peptides described herein may be composed of standard amino acids (i.e., the 20 L-alpha-amino acids that are specified by the genetic code, optionally further including selenocysteine and/or pyrrolysine). Polypeptides and peptides may comprise one or more non-standard amino acids. Non-standard amino acids can be amino acids that are found in naturally occurring polypeptides, e.g., as a result of post-translational modification, and/or amino acids that are not found in naturally occurring polypeptides. Polypeptides and peptides may comprise one or more amino acid analogs known in the art can be used. Beta-amino acids or D-amino acids may be used. One or more of the amino acids in a polypeptide or peptide may be modified, for example, by the addition of a chemical entity such as a carbohydrate group, a phosphate group, a fatty acid group, a linker for conjugation, functionalization, etc. A polypeptide that has a nonpolypeptide moiety covalently or noncovalently associated may still be referred to as a “polypeptide”. Polypeptides may be purified from natural sources, produced in vitro or in vivo in suitable expression systems using recombinant DNA technology, synthesized through chemical means such as conventional solid phase peptide synthesis and/or using methods involving chemical ligation of synthesized peptides. The term “polypeptide sequence” or “amino acid sequence” as used herein can refer to the polypeptide material itself and/or to the sequence information (i.e. the succession of letters or three letter codes used as abbreviations for amino acid names) that biochemically characterizes a polypeptide. Polypeptide sequences herein are presented in an N-terminal to C-terminal direction unless otherwise indicated.

“Variant” refers to any polypeptide or peptide differing from a naturally occurring polypeptide by amino acid insertion(s), deletion(s), and/or substitution(s), created using, e g., recombinant DNA techniques. In some embodiments amino acid “substitutions” are the result of replacing one amino acid with another amino acid having similar structural and/or chemical properties, i.e., conservative amino acid replacements. “Conservative” amino acid substitutions may be made on the basis of similarity in any of a variety or properties such as side chain size, polarity, charge, solubility, hydrophobicity, hydrophilicity, and/or amphipathicity of the residues involved. For example, the non-polar (hydrophobic) amino acids include alanine, leucine, isoleucine, valine, glycine, proline, phenylalanine, tryptophan and methionine. The polar (hydrophilic), neutral amino acids include serine, threonine, tyrosine, asparagine, and glutamine. The positively charged (basic) amino acids include arginine, lysine and histidine. The negatively charged (acidic) amino acids include aspartic acid and glutamic acid. In some embodiments cysteine is considered a non-polar amino acid. In some embodiments Insertions or deletions may range in size from about 1 to 20 amino acids, e.g., 1 to 10 amino acids. In some instances larger domains may be removed without substantially affecting function. In certain embodiments, the sequence of a variant can be obtained by making no more than a total of 1, 2, 3, 5, 10, 15, or 20 amino acid additions, deletions, or substitutions to the sequence of a naturally occurring polypeptide. In some embodiments, not more than 1%, 5%, 10%, or 20% of the amino acids in a polypeptide or fragment thereof are insertions, deletions, or substitutions relative to the original polypeptide. In some embodiments, guidance in determining which amino acid residues may be replaced, added, or deleted without eliminating or substantially reducing activities of interest, may be obtained by comparing the sequence of the particular polypeptide with that of orthologous polypeptides from other organisms and avoiding sequence changes in regions of high conservation or by replacing amino acids with those found in orthologous sequences since amino acid residues that are conserved among various species may more likely be important for activity than amino acids that are not conserved.

Certain of the inventive methods provided herein can be used to identify sequences within biofilm-forming polypeptides that mediate their assembly. In some embodiments the polypeptide is from a bacterial strain that is resistant to one or more antibiotics. Other methods can be used to identify compounds that modulate, e.g., inhibit, formation or maintenance of aggregates that contribute to biofilm formation. For example, the invention provides methods to identify peptides within CsgB that mediate assembly of CsgA into fibers comprised at least in part of CsgA, e.g., fibers comprising CsgA and CsgB. The invention also provides methods to identify peptides within CsgA that mediate assembly of CsgA into fibers comprised at least in part of CsgA. The invention further provides methods to identify peptides within CsgB that mediate assembly of CsgB into fibers comprised at least in part of CsgB. Without limiting the invention in any way, peptides within CsgB that mediate assembly of CsgA, e.g., under conditions in which peptides within CsgA do not mediate assembly of CsgA or do so much less efficiently (e.g., requiring significantly longer time such as 5, 10, 20, 50 timesas long, or longer, to achieve equivalent assembly) are of particular interest since assembly using such peptides and conditions mimics the natural process of curli fiber assembly wherein CsgB seeds assembly of CsgA.

The invention provides collections of peptides, arrays, methods of using the peptides and arrays, and related compositions and methods disclosed herein, wherein the first polypeptide is a CsgA polypeptide. The invention also provides collections of peptides, arrays, methods of using the peptides and arrays, and related compositions and methods disclosed herein, wherein the first polypeptide is a CsgB polypeptide. The invention provides collections of peptides whose sequence comprises a portion of a CsgA polypeptide sequence (“CsgA peptides”). The invention further provides collections of peptides whose sequence comprises a portion of CsgB polypeptide sequence (“CsgB peptides”), In certain embodiments, in addition to a portion of a CsgA or CsgB sequence, the peptides further comprise one or more additional amino acids, e.g., one or more alanine or lysine residues (e.g., a double alanine tag, a double lysine tag, etc.), which may be located at the N- or C-terminus of the CsgA or CsgB sequence. Without limitation, such additional residues may be useful for synthesizing the peptides or attaching the peptides to a surface. The invention provides arrays that comprise a plurality of different CsgA peptides (“CsgA peptide arrays”). The invention further provides arrays comprising a plurality of different CsgB peptides (“CsgB peptide arrays”). The invention further provides a composition comprising a CsgA peptide array and soluble CsgA. The invention further provides a composition comprising a CsgB peptide array and soluble CsgB. The invention further provides a composition comprising a CsgB peptide array and soluble CsgA. The soluble polypeptides can comprise a detectable moiety, e.g., a fluorescent or luminescent moiety such as those described above.

The invention provides compositions comprising any of the foregoing peptide collections and peptide arrays and further comprising a liquid medium. The liquid medium is, in some embodiments, one in which CsgA assembly can occur in the presence of an appropriate seeding polypeptide. The composition, in some embodiments, further comprises a CsgA polypeptide. In some embodiments the composition further comprises an amyloid-specific moiety that serves as an indicator of fiber assembly. In accordance with the invention, fibers assemble at locations on the array comprising peptides that nucleate fiber assembly. Peptide arrays having a fiber attached thereto are an aspect of the invention, wherein the fiber comprising a CsgA polypeptide. The fiber is assembled at a location where a peptide capable of seeding fiber formation is located. The fibers can be detected using, e.g., an amyloid-specific moiety or based on a detectable moiety in the polypeptide (e.g., a fluorescent label). Presence of fibers at particular locations where peptides of known identity are positioned serves to identify the peptides that nucleate assembly. Alternately, the identity of the peptides at particular locations need not be known in advance, Instead, peptides located at the positions where fibers assemble could be recovered and their sequence determined, e.g., by sequencing.

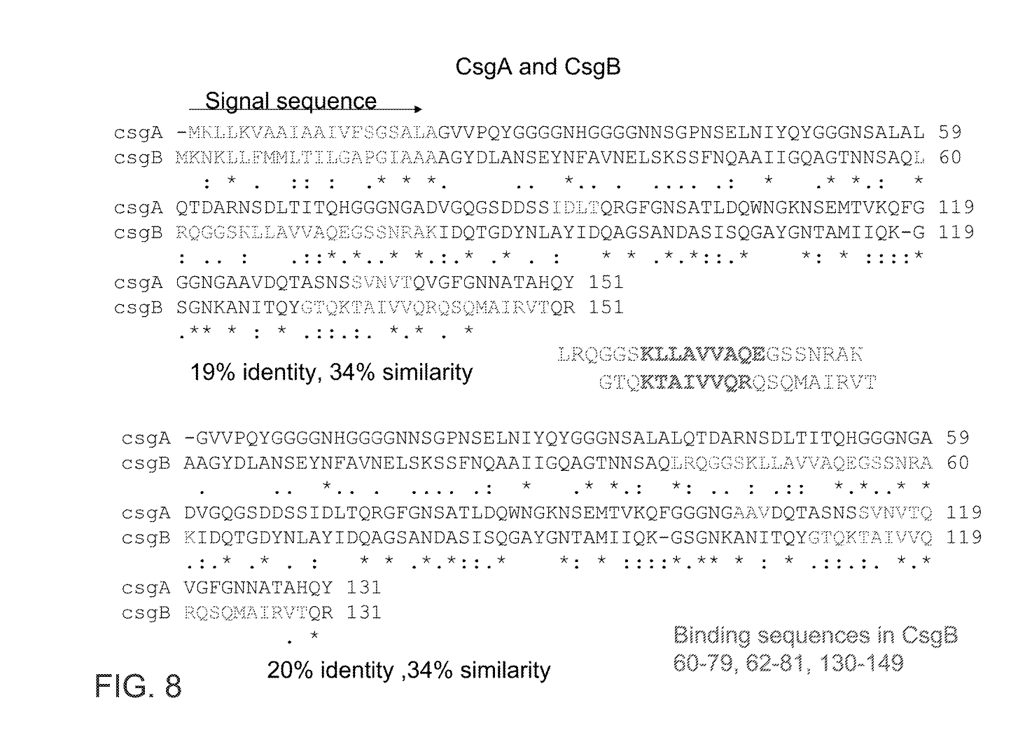

FIGS. 3A-3C show certain CsgA and CsgB sequences of use in the present invention and accession numbers thereof. Aggregation domains of CsgB polypeptides are identified using the methods provided herein. Peptides of interest comprise or consist of these sequences or portions thereof capable of nucleating aggregation of CsgA. It will be appreciated that peptides of interest can, in certain embodiments, encompass the minimal nucleating sequences and additional sequences on one or both ends. Exemplary peptides have a sequence that comprises or consists of a sequence falling within amino acids 50-90 or 120-160 of E. coli CsgB, or within the corresponding amino acids within CsgB from other bacterial species. Amino acid numbering is considered to start with the first amino acid of the full length sequence, including the signal peptide but omitting the 9 amino acid N-terminal amino acids found in some CsgB polypeptides. For example, numbering is as shown in FIG. 8. [Please note that the “ruler” shown in FIG. 3C may be shifted by 9 amino acids to the right in order to correspond with the numbering of FIG. 8 and the identity of the sequences disclosed herein.] Exemplary sequences include amino acids 55-75 or 125-155 of CsgB, or a portion of the afore-mentioned sequences. Specific examples of 25 amino acid peptides include, e.g., peptides having the sequence of amino acids 57-81, 58-82, 59-83, 60-84, 61-85, 62-86, 63-87, 125-149, 126-150, 127-151, 128-152, 129-153, 130-154, etc., of CsgB. Specific examples of 23 amino acid peptides include, e.g., peptides having the sequence of amino acids 58-80, 59-81, 60-82, 61-83, 62-84, 63-87, 127-149, 128-150, 129-151, 130-152, 131-153, 132-154, etc., of CsgB. Specific examples of 22 amino acid peptides include, e.g., peptides having the sequence of amino acids 59-80, 60-81, 61-82, 62-83, 129-150, 130-151, 131-152, etc., of CsgB. Specific examples of 21 amino acid peptides include, e.g., peptides having the sequence of amino acids 59-79, 60-80, 61-81, 62-82, 129-149, 130-150, 131-151, etc., of CsgB. Specific examples of 20 amino acid peptides include, e.g., peptides having the sequence of amino acids 60-79, 61-80, 62-81, 130-149, 131-150, etc., of CsgB.

The following peptides are exemplary: (i) LRQGGSKLLAVVAQEGSSNRAK (SEQ ID NO: 1) (CsgB 60-81); (ii) GTQKTAIVVQRQSQMAIRVT (SEQ ID NO: 2) (CsgB 130-149). In some embodiments a peptide comprises at least AIVVQ (SEQ ID NO: 3) and, optionally, one or more additional amino acids found in CsgB at locations N- or C-terminal to AIVVQ (SEQ ID NO: 3). In some embodiments a peptide comprises at least LAVVAQ (SEQ ID NO: 4) and, optionally, 1, 2, 3, 4, 5, 6, or more additional amino acids found in CsgB at locations N- or C-terminal to LAVVAQ (SEQ ID NO: 4), i.e., the peptide could be extended in either or both directions. For example, one such peptide is GGSKLLAVVAQEGSSN (SEQ ID NO: 5). Peptides can comprise KLLAVVAQE (SEQ ID NO: 6) or KTAIVVQR (SEQ ID NO: 7) and, optionally, one or more additional amino acids found in CsgB at locations N- or C-terminal to such peptides, i.e., the peptide could be extended in either or both directions by, for example, 1, 2, 3, 4, 5, or 6 amino acids. For example, one such peptide is TQKTAIVVQRQSQMAIR (SEQ ID NO: 8). In some embodiments a peptide is between 5 and 25 amino acids long, e.g., 6, 7, 8, 9, 10, 11, 12, 13, 14, 15, 16, 176, 18, 19, 20, 21, 22, 23, 24, or 25 amino acids long.

It will be appreciated that SEQ ID NOs: 1-8 are found in certain E. coli strains. Minor differences may be encountered in other E. coli strains or in CsgB polypeptides from different bacterial genera. Peptides that are orthologs of the afore-mentioned peptides in any particular bacterial strain, species, genus, or family are provided. One of skill in the art will be able to identify such orthologs based on sequence comparisons. Also provided are variants of any of the afore-mentioned peptides. In some embodiments, a variant of a particular peptide may have 1, 2, or 3 amino acid substitutions, additions, and/or deletions relative to the original peptide. In some embodiments a substitution is a conservative substitution. In some embodiments a polar or hydrophilic amino acid is added or substituted. Optionally the peptides further comprise a tag, detectable moiety, etc. Peptides may be tested using the methods described herein to select those that may be preferable for use in any particular method, e.g., for performing screens, detecting presence of bacteria, etc. The optimal peptide may differ depending on various factors such as the conditions of the assay, the particular bacteria to be detected, etc. Each of the peptides described herein is an aspect of the invention. The various aspects of the invention include embodiments that relate specifically to each of these peptides. For example, the invention provides antibodies that bind to each of these peptides, methods of using each of the peptides as a vaccine component, methods of designing inhibitors of biofilm formation or maintenance and/or amyloid assembly based on each of the peptides, etc. The peptides can also be used to identify the precise amino acids within CsgA that mediate curli assembly, For example, the CsgB peptides disclosed herein can be used to identify those amino acids within a CsgA polypeptide that form contacts with the peptides. The invention further provides compositions containing any one or more of the peptides, wherein the peptide is at least partly purified or synthetically produced. In some embodiments a composition further comprises a CsgA, CsgC, CsgD, CsgE, CsgF, and/or CsgB polypeptide.

Also provided are polypeptides and peptides that comprise any of the foregoing peptides of SEQ ID NO: 1-8, wherein the peptide of SEQ ID NO: 1-8 is not found in the same amino acid context as when present in CsgB.

The methods provided herein can be used to screen for agents that inhibit biofilm formation or maintenance and/or that disrupt biofilms that have already formed. Such agents could be used as components of washes or disinfectant solutions (e.g., in combination with a suitable carrier such as water), to impregnate cleaning supplies such as sponges, wipes, or cloths, or as components of surface coatings (e.g., in combination with a suitable carrier such as a polymeric material) for a variety of medical devices. They could be added to existing disinfectant or anti-microbial compositions. In certain embodiments they are used as prophylactic or therapeutic agents in individuals who are susceptible to infection, infected (e.g., by biofilm-forming bacteria), and/or have an indwelling or implantable device, are immunocompromised (e.g., individuals suffering from HIV, individuals taking immunosuppressive medication, individuals with immune system deficiencies or dysfunction) are hospitalized, are less than 6 weeks old, less than 2 years old, over 65 years of age, have an implanted prosthetic or medical device (e.g., an artificial heart valve, joint, stent, orthopedic appliance, etc.). Biofilms are often associated with cystic fibrosis, endocarditis, osteomyelitis, otitis media, urinary tract infections, oral infections, and dental caries, among other conditions. In some instances a biofilm-associated infection is a nosocomial infection. In some cases a biofilm-associated infection is a mixed infection, comprising multiple different microorganisms. In some cases an individual suffering from a biofilm-associated infection is at increased risk of contracting a second infection.

In some embodiments, the agent is used as a component of a coating for a catheter, stent, valve, pacemaker, conduit, cannula, appliance, scaffold, central line, IV line, pessary, tube, drain, trochar or plug, implant, a rod, a screw, or orthopedic or implantable prosthetic device or appliance. In another embodiment, the agent is used as a component of a coating for a conduit, pipe lining, a reactor, filter, vessel, or equipment which comes into contact with a beverage or food, e.g., intended for human or animal consumption or treatment, or water or other fluid intended for consumption, cleaning, agricultural, industrial, or other use. In some embodiments the agent is used as a component of a wound dressing, bandage, toothpaste, cosmetic, etc.

A surface having a CsgB peptide that nucleates curli fiber formation attached thereto can serve as a sensor for the presence of curli-producing bacteria. Peptides that specifically mediate assembly of CsgA and/or CsgB polypeptides from different bacteria could be deposited on a surface. The surface is placed in a fluid or medium that is to be tested. In some embodiments, curb fiber formation is detected. For example, after incubating the surface in the medium to be tested, the surface is contacted with an amyloid indicator substance such as Congo Red. In some embodiments, the peptide “concentrates” the bacteria by facilitating biofilm assembly. Following a suitable time period the surface is “stamped” onto culture plates. Growth or presence of curli fibers at a specific position on the plate is correlated with the sequence of the peptide located at a particular position on the surface, thereby identifying the bacteria. Alternately the bacteria may be identified using a suitable bacterial identification method.

Methods provided herein may be used to capture and/or detect a CsgA polypeptide without use of antibodies, aptamers, cross-linking agents, etc.

In another embodiment a surface having a peptide, e.g., a CsgB peptide that nucleates curli fiber formation attached thereto, is used to remove CsgA and/or CsgB polypeptides from a solution. The solution may be, e.g., water or a body fluid such as blood, plasma, serum, etc. The fluid is contacted with the surface under conditions suitable for aggregate assembly. After a suitable period of time polypeptides present in the solution aggregate on the surface and can thus be efficiently removed. In one embodiment, such a method is used to treat a subject either ex vivo or in vivo. In one embodiment the surface is used to remove polypeptides from a blood product to be administered to a subject. In one embodiment the surface is used to treat an organ to be transplanted into a subject. The organ may be bathed in a solution containing an inventive peptide prior to transplantation. In some embodiments peptides are attached to particles, also referred to as “beads”. The beads may be magnetic. In one embodiment the method is used to remove polypeptides from a body fluid in a subject undergoing dialysis. In some embodiments peptides are attached to beads that are administered to a subject. The beads may be composed of a biocompatible material, e.g., a biodegradable material.

In certain embodiments of the inventive methods, a plurality of ˜20-mer peptides having a sequence that comprises at least 8 and no more than 50 amino acids of the sequence of a first amyloidogenic polypeptide such as a CsgB polypeptide, each including a double lysine tag attached by a PEG linker are attached at their C-terminal ends to a cellulose membrane. The peptides are cleaved from the membrane and printed on a reactive glass slide (e.g., an aldehyde functionalized glass slide with 3×300-1000 spots per slide). In one embodiment, peptide density is about 15-150 fmol/mm². The slide is blocked for about 1 hr in 3% BSA, 0.1% T₂O. Denatured second amyloidogenic polypeptide (e.g., CsgA) is prepared and diluted into PBS buffer. At least some of the polypeptide is labeled, e.g., with ALEXA FLUOR® 532 or ALEXA FLUOR® 647. For example, about 5% of the polypeptides may be labeled. The slide is placed in the chamber and the polypeptides in solution (e.g., CsgA polypeptides) are allowed to hybridize without rotation. The array is then removed from the chamber and washed with 2% SDS. The array is subsequently imaged at appropriate wavelengths to detect aggregate formation that takes place on features that have peptides containing a nucleating sequence attached thereto.

The invention encompasses numerous variations of the above method. For example, the peptides can be synthesized using any convenient method. The peptides need not be deposited on a surface. In some embodiments the peptides are deposited in individual vessels, e.g., wells of a microwell plate. In some embodiments the peptides are placed in liquid medium in individual vessels. It will be appreciated that details such as buffers, blocking reagents, washing steps, etc., can be varied. In some embodiments, the polypeptide in solution is detectably labeled. For example, the polypeptide may have an optically detectable moiety attached thereto. In some embodiments the polypeptide in solution does not have an optically detectable moiety attached thereto. In some embodiments the polypeptide in solution is denatured. In some embodiments the polypeptide is not denatured. In some embodiments, aggregation of the polypeptide is detected by including in the assay system a substance that binds to protein aggregates and may be used to detect them. The substance may undergo a change in optical properties upon binding.

Methods for identifying an agent for modulating protein aggregation, e.g., enhancing or inhibiting or altering the kinetics of protein aggregation are provided herein. One such method comprises steps of: (a) providing a composition that comprises (i) a peptide derived from a first amyloidogenic polypeptide; (ii) a second amyloidogenic polypeptide that binds to the peptide in the absence of the test agent; and (iii) a test agent; and (b) identifying the test agent as a candidate agent for modulating protein aggregation if presence of the test agent alters the extent or rate of binding of the peptide and the polypeptide. Another such method includes: (a) providing a composition that comprises (i) a peptide derived from a first amyloidogenic polypeptide; (ii) a second amyloidogenic polypeptide wherein the peptide is capable of seeding aggregation of the second polypeptide in the absence of the test agent; and (iii) a test agent; and (b) identifying the test agent as a candidate agent for modulating protein aggregation if presence of the test agent alters the extent or rate of aggregate formation.

Methods for identifying an agent for inhibiting protein aggregation are provided herein. One such method comprises: (a) providing a composition that comprises (i) a peptide derived from a first amyloidogenic polypeptide; (ii) a second amyloidogenic polypeptide that binds to the peptide in the absence of the test agent; and (iii) a test agent; and (b) identifying the test agent as an agent for inhibiting protein aggregation if presence of the test agent reduces the binding of the peptide and the polypeptide. The first and second amyloidogenic polypeptides may be CsgB and CsgA. Another such method comprises: (a) providing a composition that comprises (i) a peptide derived from a first amyloidogenic polypeptide; (ii) a second amyloidogenic polypeptide that binds to the peptide in the absence of the test agent; and (iii) a test agent; and (b) identifying the test agent as an agent for inhibiting protein aggregation if presence of the test agent reduces aggregation of the polypeptide. In some embodiments of these methods the first amyloidogenic polypeptide is CsgA or CsgB and the second polypeptide is a polypeptide whose aggregation is associated with mammalian disease, e.g., serum amyloid A protein.

The peptide may be any peptide identified according to the methods for identifying aggregation domains described herein. The peptide is usually at least 5 amino acids long, e.g., 5-8, 8-10, 10-15, 15-20, 20-25 amino acids long. A peptide is “derived from” a polypeptide if it has or comprises the same sequence as a portion of the polypeptide or, in some embodiments is at least 80%, at least 90%, or at least 95% identical to a portion of the polypeptide over its length. The percent identity between a sequence of interest and a second sequence over a window of evaluation may be computed by aligning the sequences, determining the number of residues (amino acids) within the window of evaluation that are opposite an identical residue (optionally allowing the introduction of gaps to maximize identity), dividing by the total number of residues of the sequence of interest or the second sequence (whichever is greater) that fall within the window, and multiplying by 100. When computing the number of identical residues needed to achieve a particular percent identity, fractions are to be rounded to the nearest whole number. Percent identity can be calculated with the use of a variety of computer programs known in the art. For example, computer programs such as BLAST2, BLASTN, BLASTP, Gapped BLAST, etc., generate alignments and provide percent identity between sequences of interest. In some embodiments % identity is determined permitting introduction of gaps while in other embodiments not permitting the introduction of gaps.

In one embodiment of the method, the polypeptide and the peptide are contacted with one another in the absence of the test agent under conditions suitable for binding and are allowed to bind. The test agent is then added, and its ability to disrupt aggregates is assessed. In one embodiment, the polypeptide and the peptide are contacted with one another in the absence of the test agent under conditions suitable for binding and the test agent is added a short time thereafter, e.g., before substantial binding has occurred. The ability of the test agent to inhibit aggregate formation is assessed. Standard methods of assessing complex formation or disruption can be employed. For example, the aggregates can be imaged and/or detection based on mass or alteration in other physical properties can be used. The polypeptide can be labeled, e.g., with a fluorescent or luminescent moiety to facilitate detection of aggregates. In some embodiments, at least some of the polypeptides comprise a moiety capable of producing a detectable signal or a moiety capable of quenching a detectable signal. Exemplary moieties include dye fluorophores, quenchers, inorganic materials such as metal chelates, metal and semiconductor nanocrystals (e.g., “quantum dots”), and fluorophores of biological origin such as fluorescent proteins and amino acids; and biological compounds that exhibit bioluminescensce upon enzymatic catalysis. Specific examples include acridine dyes; Alexa dyes; BODIPY, cyanine dyes; fluorescein and derivatives thereof; rhodamine derivatives thereof; green, blue, sapphire, yellow, red, orange, and cyan fluorescent proteins and derivatives thereof; monomeric red fluorescent protein (mRFP1) and derivatives such as those known as “mFruits”, e.g., mCherry, mStrawberry, etc. Organic UV dyes include a variety of pyrene, naphthalene, and coumarin-based structures. Visible/near IR dyes include a number of fluorescein, rhodamine, and cyanine-based derivatives. Quenchers include dabsyl (dimethylaminoazosulphonic acid), Black Hole® quenchers (Biosearch Technologies), Qxl® quenchers (AnaSpec). In some embodiments, at least some of the polypeptides comprise a first moiety that generates or quenches a signal, and at least some of the polypeptides are labeled with a moiety that is capable of quenching the signal. The polypeptide could include an epitope tag to facilitate detection using an enzyme-linked or otherwise detectable antibody that binds to the tag. In certain embodiments, of course, it is not necessary to use such a moiety or tag to determine whether the test agent inhibits formation of or disrupts higher order aggregates. For example, a variety of other approaches could be used, such as use of moieties that bind to aggregates. Also, it may be of interest to identify agents capable of accelerating or otherwise enhancing aggregate formation. Such agents could be of use, e.g., to increase the speed with which a screen for inhibitors can be performed, e.g., by decreasing the lag time for fiber assembly. Without wishing to be bound by any theory, inhibitors capable of inhibiting aggregation even in the presence of an enhancer of aggregation may be particularly effective inhibitors. The peptides could be in solution or attached to a support such as a glass or plastic surface (e.g., a slide, multiwall dish, tube), membrane, filter, particle (e.g., microparticles such as beads, nanoparticles, etc.), etc. In some embodiments peptides are attached to a sensor device capable of detecting binding thereto. Such devices include, e.g., sensors that utilize surface plasmon resonance to detect binding (e.g., Biacore™ systems), suspended microchannels (see, e.g., U.S. Pat. No. 7,282,329), cantilever-based systems and others that detect changes in mass upon binding, etc.

A variety of different test agents can be tested using the inventive methods. A test agent can be any molecule or supramolecular complex, e.g. polypeptides, peptides, small organic or inorganic molecules (i.e., molecules having a molecular weight less than 2,500 Da, 2000 Da, 1,500 Da, 1000 Da, or 500 Da in size, and in some embodiments at least 50 Da, at least 100 Da, at least 150 Da in size), polysaccharides, polynucleotides, etc. which is to be tested for ability to modulate aggregate formation or disrupt aggregates that have already formed. In some embodiments, the test agents are organic molecules, particularly small organic molecules, including functional groups that mediate structural interactions with proteins, e.g., hydrogen bonding, and typically include at least an amine, carbonyl, hydroxyl or carboxyl group, and in some embodiments at least two of the functional chemical groups. The test agents may include cyclic carbon or heterocyclic structures and/or aromatic or polyaromatic structures substituted with one or more chemical functional groups and/or heteroatoms. Test agents may be obtained from a wide variety of sources, as will be appreciated by those in the art, including libraries of synthetic or natural compounds.

In some embodiments, test agents are synthetic compounds. Numerous techniques are available for the random and directed synthesis of a wide variety of organic compounds and biomolecules. In some embodiments, the test modulators are provided as mixtures of natural compounds in the form of bacterial, fungal, plant and animal extracts, fermentation broths, etc., that are available or readily produced. In some embodiments, a library of compounds is screened. The term “library of compounds” is used consistently with its usage in the art. A library is typically a collection of compounds that can be presented or displayed such that the compounds can be identified in a screening assay. In some embodiments compounds in the library are housed in individual wells (e.g., of microtiter plates), vessels, tubes, etc., to facilitate convenient transfer to individual wells or vessels for contacting cells, performing cell-free assays, etc. The library may be composed of molecules having common structural features which differ in the number or type of group attached to the main structure or may be completely random. Libraries include but are not limited to, for example, phage display libraries, peptide libraries, polysome libraries, aptamer libraries, synthetic small molecule libraries, natural compound libraries, and chemical libraries. Methods for preparing libraries of molecules are well known in the art and many libraries are available from commercial or non-commercial sources. Libraries of interest include synthetic organic combinatorial libraries. Libraries, such as synthetic small molecule libraries and chemical libraries, can include a structurally diverse collection of chemical molecules. Small molecules include organic molecules often having multiple carbon-carbon bonds. The libraries can include cyclic carbon or heterocyclic structure and/or aromatic or polyaromatic structures substituted with one or more functional groups. In some embodiments the small molecule has between 5 and 50 carbon atoms, e.g., between 7 and 30 carbons. In some embodiments the compounds are macrocyclic. Libraries of interest also include peptide libraries, randomized oligonucleotide libraries, and the like. Libraries can be synthesized of peptides and non-peptide synthetic moieties. Such libraries can further be synthesized which contain non-peptide synthetic moieties which are less subject to enzymatic degradation compared to their naturally-occurring counterparts. Small molecule combinatorial libraries may also be generated. A combinatorial library of small organic compounds may include a collection of closely related analogs that differ from each other in one or more points of diversity and are synthesized by organic techniques using multi-step processes. Combinatorial libraries can include a vast number of small organic compounds. In one embodiment, the methods provided herein are used to screen approved drugs. An approved drug includes any compound (which term includes biological molecules such as proteins and nucleic acids) which has been approved for use in humans by the FDA or a similar government agency in another country, for any purpose. This can be a particularly useful class of compounds to screen because it represents a set of compounds which are believed to be safe and, at least in the case of FDA approved drugs, therapeutic for at least one purpose. Thus, there is a high likelihood that these drugs will at least be safe for other purposes. Natural or synthetically produced libraries and compounds are readily modified through conventional chemical, physical and biochemical means. Chemical (including enzymatic) reactions may be done on the moieties to form new substrates or test agents which can then be tested using the methods and peptide compositions provided herein. Known pharmacological agents may be subjected to directed or random chemical modifications, including enzymatic modifications, to produce structural analogs.

In some embodiments, test agents are peptides or nucleic acids. In some embodiment, test agents are naturally occurring polypeptides or fragments of naturally occurring polypeptides, e.g., from bacterial, fungal, viral, and mammalian sources. In some embodiments, test agents are nucleic acids of from about 2 to about 50 nucleotides, e.g., about 5 to about 30 or about 8 to about 20 nucleotides in length. For example, test agents could be aptamers. In some embodiment, test modulators are peptides of from about 2 to about 60 amino acids, e.g., about 5 to about 30 or about 8 to about 20 amino acids in length. The peptides may be digests of naturally occurring polypeptides or randomly synthesized peptides that may incorporate any amino acid at any position. In some embodiments a synthetic process is used to generate randomized polypeptides or nucleic acids, to allow the formation of all or most of the possible combinations over the length of the sequence, thus forming a library of randomized candidate bioactive agents. For example, a library of all combinations of amino acids that form a peptide 7 to 20 amino acids in length could be used. In some embodiments, the library is fully randomized, with no sequence preferences, constraints, or constants at any position. In some embodiments, the library is biased, i.e., some positions within the sequence are either held constant, or are selected from a limited number of possibilities. For example, the nucleotides or amino acid residues may be randomized within a defined class, for example, of hydrophobic, hydrophilic, acidic, or basic amino acids, sterically biased (either small or large) residues, towards the creation of cysteines for cross-linking, prolines for turns, serines, threonines, tyrosines or histidines for phosphorylation sites, etc. The peptides could be cyclic or linear. It will be appreciated that the above description of test agents is indicative of some of the various compound types and classes into which agents to be tested may fall.

The invention encompasses the recognition that peptides that mediate aggregation, e.g., peptides identified according to the inventive methods, may be used to inhibit aggregation or may be modified or used as starting points to develop agents that inhibit aggregation. Such peptides may bind to a polypeptide, e.g., a CsgA polypeptide, and prevent it from being added to a growing aggregate or may bind to polypeptides within a growing aggregate and thereby inhibit binding of additional polypeptides to the aggregate. The peptide may be used to target a moiety of interest to the polypeptide or assembling aggregate. The moiety of interest could be a disrupting agent, a label, etc. The invention provides peptides containing sequences that mediate aggregation, e.g., peptides identified according to the inventive methods, linked to a disrupting agent. The disrupting agent is a moiety that inhibits or disrupts aggregate formation, e.g., fiber assembly. In some embodiments at least one end (N-terminal and/or C-terminal end) of the peptide is flanked with one or more β-strand breaking amino acids such as proline or D-amino acids which might stereospecifically block further polymerization. In some embodiments the disrupting agent comprises a sequence of polar charged amino acids, e.g., polylysine, a sequence of between 4 and 10 lysines.

The invention further provides agents that comprise a peptide that mediates aggregation, e.g., a peptide identified as described herein, wherein the peptide is covalently or noncovalently linked to an agent that inhibits fiber assembly. The agent could be one identified by screening as described herein or identified using any method known in the art. Various agents have been identified as being useful to inhibit or disrupt various amyloid aggregates. For example, staurosporine derivatives and related molecules e.g., analogs of DAPH have been shown to have such properties with regard to a number of amyloids. See, e.g., U.S. patent application Ser. No. 11/258,391. Small molecule inhibitors of polyglutamine amyloid formation have been identified (Ehrnhoefer et.al, Hum.Mol.Genet., 15; 2743, 2006). Such agents could be attached to aggregation-mediating peptides using standard methods, e.g., bioconjugation methods such as those described in Hermanson, G., et al., Bioconjugate Techniques, Academic Press; 2nd edition, 2008.

The invention further provides a library of peptides generated by modifying or randomizing one or more positions within a peptide that mediates protein aggregation, e.g., a peptide disclosed herein or any peptide identified according to the inventive methods. For example, the invention provides a library of peptides generated by modifying, e.g., randomizing one or more positions within a CsgB peptide that is capable of seeding formation of a fiber comprising CsgA polypeptide.

In some embodiments test agents are antibodies, antibody fragments, or other agents comprising an antigen binding domain of an immunoglobulin. In some embodiments the test agent is an antibody or antibody fragment generated against a peptide, wherein the peptide is an aggregation domain of a polypeptide. The antibody may be monoclonal or polyclonal and may be of any of the antibody classes, in various embodiments of the invention. Antibodies or antibody fragments having an antigen binding region, including fragments such as Fv, Fab′, F(ab′)2, Fab fragments, single chain antibodies (which include the variable regions of the heavy and light chains of an immunoglobulin, linked together with a short (usually serine, glycine) linker, polyclonal, monoclonal, chimeric or humanized antibodies, and complimentarily determining regions (CDR) may be prepared by conventional procedures. Peptides identified according to the inventive methods may be used as antigens to generate such antibodies or antibody fragments using standard methods. Such antibodies and antibody fragments are an aspect of the invention. Furthermore, peptides identified according to the inventive methods may be used as components of vaccines. The vaccines could be administered prior to or following exposure to a bacterium. In some embodiments a vaccine contains a peptide identified by the methods herein and one or more additional components such as an adjuvant, excipient, etc., as conventionally used to prepare vaccines. Vaccines could be administered to any subject (human, animal) at risk of or suffering from infection with a biofilm-forming bacterium. In some embodiments a nucleic acid construct that encodes an inventive peptide or encodes a polypeptide that comprises an inventive peptide is administered for prophylactic or therapeutic purposes. Optionally the polypeptide comprises a signal peptide so that it will be secreted, e.g., by a mammalian cell.

A test agent identified or generated using the methods provided herein may be useful for a wide variety of purposes. In certain embodiments, the test agent inhibits formation of a protein aggregate outside of cells but within a living organism. The agent may be useful for treatment or prophylaxis of a condition or disease associated with protein aggregation. The agent may also be used to regulate formation of higher order aggregates in vitro. In certain embodiments the agent is useful to treat a disease or condition associated with biofilm formation or curli fiber expression, e.g., a bacterial infection. The agent may be given prophylactically, e.g., before an individual has developed symptoms associated with infection, or after symptoms develop. In some embodiments the agent inhibits additional aggregate formation. In some embodiments aggregates that have already formed are disrupted by the agent. The agents may be used to inhibit one or more pathogenic activities associated with curli fibers and/or other bacterial amyloids. In some embodiments an agent is used to inhibit bacterial attachment to and/or invasion of host cells. In some embodiments an agent is used to inhibit interaction of bacteria or curli with host proteins such as extracellular matrix proteins (e.g., fibronectin, lamimin), H-kininogen, serine proteases such as plasminogen, tissue plasminogen activator, fibrinogen, factor XII, etc.

A test agent identified using an inventive method described herein may undergo additional testing, e.g., in a biological system comprising a living organism or organisms, to evaluate its efficacy or other properties. In some embodiments an identified agent is tested in a biological system comprising living organisms, e.g., bacteria, that have the capacity to produce amyloid. The effect of the agent on amyloid formation or maintenance is assessed. In some embodiments an identified agent is tested in a biological system comprising living organisms that are susceptible to disease associated with presence of amyloid. In some embodiments the effect of the agent on development or severity of the disease is assessed. In some embodiments the biological system comprises bacteria that have the capacity to produce amyloid. In some embodiments an identified agent is tested in a biological system comprising living organisms that are susceptible to infection with bacteria that have the capacity to produce curli. The effect of the agent on pathogenesis of the bacteria or on one or more properties of the bacteria such as cell adhesion or invasion is assessed. In some embodiments an agent identified according to the inventive methods is tested in animal models to further explore its effects on pathogenesis or biofilm formation and/or to further evaluate therapeutic potential. Animal models for a variety of infectious diseases associated with amyloid-producing bacteria and/or biofilm formation are known.

When administered to a human or animal subject, an agent identified according to the invention can be administered as a pharmaceutical composition comprising a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil or injectable organic esters. A pharmaceutically acceptable carrier can contain physiologically acceptable compounds that act, for example, to stabilize or to increase the absorption of the active therapeutic compound. The physiologically acceptable compounds include, for example, carbohydrates, such as glucose, sucrose or dextrans, antioxidants, such as ascorbic acid or glutathione, chelating agents, buffers, low molecular weight proteins or other stabilizers or excipients. One skilled in the art would know that the choice of a pharmaceutically acceptable carrier, including a physiologically acceptable compound, depends, for example, on the route of administration of the composition. The pharmaceutical composition could be in the form of a liquid, gel, lotion, tablet, capsule, ointment, etc. One skilled in the art would know that a pharmaceutical composition can be administered to a subject by various routes including, for example, oral administration; intramuscular administration; intravenous administration; anal administration; vaginal administration; parenteral administration; nasal administration; intraperitoneal administration; subcutaneous administration and topical administration. One skilled in the art would select an effective dose and administration regimen taking into consideration factors such as the patient's weight and general health, the particular condition being treated, etc.

The pharmaceutical composition can also be delivered by means of a microparticle or nanoparticle or a liposome or other delivery vehicle or matrix. A number of biocompatible polymeric materials are known in the art to be of use for drug delivery purposes. Examples include polylactide-co-glycolide, polycaprolactone, polyanhydride, and copolymers or blends thereof.

A peptide or other agent identified using an inventive method could be administered in combination with other agents useful for prophylactic purposes and/or to treat an existing infection, either in the same composition or individually. Such agents could be, e.g., any suitable anti-infective, e.g., antibacterial or antifungal agents, etc. Examples include, but are not limited to, amikacin, gentamicin, tobramycin, amoxicillin, amoxicillin/clavulanate, amphotericin B, ampicillin, ampicillin/sulbactam, atovaquone, azithromycin, cefazolin, cefepime, cefotaxime, cefotetan,cefpodoxime, ceftazidime, ceftizoxime, ceftriaxone, cefuroxime, cephalexin, chloramphenicol, clotrimazole, ciprofloxacin, clarithromycin, clindamycin, cicloxacillin, coxycycline, echincandins, erythromycin (including estolate, ethylsuccinate, gluceptate, lactobionate, and stearate), famciclovir, fluconazole, foscarnet, ganciclovir, imipenem/cilastatin (Primaxin), isoniazid, itraconazole, ketoconazole, metronidazole, nafcillin, nitrofurantoin, nystatin, penicillin (including G benzathine, G potassium, G procaine, V potassium), pentamidine, piperacillinitazobactam, rifampin, ticarcillin/clavulanate, trimethoprim, trimethoprim sulfate, valacyclovir, vancomycin, aztreonam, levofloxacin, meropenem, tobramycin, cephalothin, mezlocillin, nalidixic acid, netilmicin, minocycline, ofloxacin, norfloxacin, sulfamethoxazole, tetracycline, neomycin, streptomycin, ticarcillin, carbenicillin, cloxacillin, cefoxitin, ceforanide, teicoplanin, ristocetin, viomycin, capreomycin, bacitracin, gramicidin, gramicidin S, tyrocidine, tachyplesin, kanamycin, methicillin, oxacillin, azocillin, bacampicillin, carbenicillin indanyl, cephapirin, cefaxolin, cephradine, cefradoxil, cefamandole, cefaclor, cefuromime axetil, cefonicid, cefoperazone, demeclocytetracycline, methacycline, oxytetracycline, spectinomycin, ethambutol, aminosalicylic acid, pyrazinamide, ethionamide, cycloserine, dapsone, sulfoxone sodium, clofazimine, sulfanilamide, sulfacetamide, sulfadiazine, sulfixoxazole, cinoxacin, methenamine, phenazopyridine, and various human or animal antibacterial peptides such as defensins, magainins, cathelidicins, or histatins. An inventive peptide or agent could also be administered or used together with other agents or strategies for inhibiting bioflim formation or maintenance.

Certain compositions and methods provided herein are of use to build structures of a desired shape and composition, which are also an aspect of the invention. Peptides, e.g., a CsgB peptide disclosed herein, can be deposited or synthesized on a surface in a desired pattern and combination. The surface is then contacted with a solution containing one or more compatible polypeptide(s), e.g., a CsgA polypeptide. The polypeptides assemble to form higher ordered aggregates at the positions where the peptide that induces assembly is located on the surface. In some embodiments the structures are nanostructures. Such structures may have at least one dimension, e.g., height, width, length, less than 1 μm. In some embodiments a conductive or resistive substance, e.g., a suitable metal, polymer, or ceramic material, is deposited on the structure. In some embodiments the structure consists of at least 25%, 50%, 75%, 90%, 95% or more polypeptide, e.g., CsgA and/or CsgB polypeptide by weight.

In another aspect, the invention provides a compound that comprises a protein aggregation domain, e.g., a CsgB peptide disclosed herein, linked to a moiety of interest. The moiety of interest may be or comprise, e.g., a peptide, a protein, a polynucleotide, a sugar, a tag, a metal atom, a particle (e.g., a nanoparticle or microparticle), a catalyst, a non-polypeptide polymer, a specific binding element (e.g., biotin, avidin or streptavidin, an antibody or antibody fragment comprising an antigen-binding domain), a small molecule, a lipid, or a label. The linkage could be covalent or noncovalent. In some embodiments the protein aggregation domain is directly linked to the moiety while in other embodiments the protein aggregation domain and the moiety are each linked to a third moiety, which serves as a linking moiety.

In another aspect, the invention provides a chimeric polypeptide that comprises a protein aggregation domain described herein, e.g., a CsgB peptide, and a polypeptide of interest. In some embodiments the chimeric polypeptide is a fusion protein. The protein aggregation domain may be located N-terminal or C-terminal to the polypeptide of interest. The polypeptide of interest can be any polypeptide that is of interest from a commercial, research, or practical standpoint. Exemplary polypeptides of interest include: enzymes that may have utility in chemical, food-processing (e.g., amylases), biofuel production, waste treatment, or other commercial applications; enzymes having utility in biotechnology applications, including DNA and RNA polymerases, endonucleases, exonucleases, peptidases, and other DNA and protein modifying enzymes; polypeptides that are capable of specifically binding to compositions of interest, such as polypeptides that act as intracellular or cell surface receptors for other polypeptides, for steroids, for carbohydrates, or for other biological molecules; polypeptides that include at least one antigen binding domain of an antibody; polypeptides that include the ligand binding domain of a ligand binding protein (e.g., the ligand binding domain of a cell surface receptor); metal binding proteins (e.g., ferritin (apoferritin), metallothioneins, and other metalloproteins), which are useful for isolating/purifying metals from a solution containing them for metal recovery or for remediation of the solution; light-harvesting proteins (e.g., proteins used in photosynthesis that bind pigments); proteins that can spectrally alter light (e.g., proteins that absorb light at one wavelength and emit light at another wavelength); regulatory proteins, such as transcription factors and translation factors; and polypeptides of therapeutic value, such as chemokines, cytokines, interleukins, growth factors, interferons, antibiotics, immunopotentiators and immunosuppressors, and angiogenic or anti-angiogenic peptides, marker proteins such as a fluorescent protein (e.g., green fluorescent protein or firefly luciferase), an antibiotic resistance-conferring protein, a protein involved in a nutrient metabolic pathway that confers selective growth on incomplete growth media, or a protein (e.g. β-galactosidase, an alkaline phosphatase, or a horseradish peroxidase) involved in a metabolic or enzymatic pathway that acts on a chromogenic or luminescent substrate to produce a detectable chromophore or light signal that can be used for identification, selection, or quantitation, proteins (e.g., glutathione S-transferase or Staphylococcal nuclease) that are used in the art as fusion partners for the purpose of facilitating expression or purification of other proteins. Also provided are nucleic acids that encode any of the peptides or polypeptides disclosed herein. Also provided are expression vectors comprising any of the nucleic acids that encode a peptide or polypeptide disclosed herein. Expression vectors typically contain a nucleic acid sequence that codes for the peptide or polypeptide, operably linked to a promoter capable of directing expression in a host cell of interest. In some embodiments the promoter is inducible (e.g., by an inducer such as a small molecule, metal, or condition such as heat). In some embodiments the promoter is constitutive. Also provided are host cells (e.g., bacterial, fungal, insect, mammalian cells) that contain or express any of the nucleic acids that encode a peptide or polypeptide disclosed herein.

### EXAMPLES

Materials and Methods

Peptide array synthesis, hybridization and quantification. The peptides are synthesized on modified cellulose membranes using SPOT™ technology (JPT Peptide Technologies GmbH) (Frank, R. Spot-Synthesis—an Easy Technique for the Positionally Addressable, Parallel Chemical Synthesis on a Membrane Support. Tetrahedron 48, 9217-9232 (1992)). Each peptide contains a double alanine tag at its N-terminus, 20 residues from CsgB, a hydrophilic linker (1-amino-4,7,10-trioxa-13-tridecanamine succinimic acid (Zhao, Z. G., Im, J. S., Lam, K. S. & Lake, D. F. Site-specific modification of a single-chain antibody using a novel glyoxylyl-based labeling reagent. 10, 424-430 (1999)) and a double lysine tag at its C-terminus. The peptides are cleaved off the membranes, freeze dried and resuspended in buffer (40% DMSO, 5% glycerol, 55% PBS, pH 9) for printing. The peptides were then printed onto hydrogel glass slides (NEXTERION® Slide H, Schott) functionalized with reactive NHS ester moieties. Each peptide spot (250 μM in diameter) is printed with 3 drops of 0.5 nL of peptide solution at a concentration of approximately 2.5 μM using non-contact printing (JPT Peptide Technologies GmbH). The unreacted peptides are removed from the hydrogel slides, dried and then the slides are blocked with 3% BSA in PBST for 1 hr. CsgA proteins are denatured in 6 M GuHCl at 100° C. for approximately 20 minutes, diluted 125 times in PBST containing 3% BSA to a final concentration of 1-5 μM and a label ratio of 5-75%. A single peptide array is incubated with approximately 2-3 mL of diluted CsgA using an ATLAS™ hybridization chamber (BD Biociences) without mixing for a given period of time. The peptide arrays are then washed 5 times with 50 mL of 2% SDS for 30 minutes, 5 times with 50 mL of water, 3 times with 50 mL of methanol and then spun dry. The methanol washes are not essential but help prevent uneven drying of the slides. The arrays are imaged using a GENEPIX® 4000A scanner and the median values for the peptide spots of two to three replicates are quantified using GENEPIX® Pro 6.0 software (Molecular Devices).

In vitro nucleation and seeding assays. Unseeded and seeded reactions are performed using either a Molecular Devices SPECTRAMAX® M2 or a Tecan SAFIRE²™ plate reader. For unseeded reactions black microtiter plates (MICROFLUOR1®, Thermo Labsystems) containing a single 4 mm glass bead per well are blocked with 2.5 mg/mL of ovalbumin in PBS with 40 μM Thioflavin T (ThT) for several hours. After the blocking solution is removed, denatured CsgA is diluted to 4 μM in PBS containing 40 μM ThT and loaded into the microtiter plate. Each plate is mixed for 10 sec/min and the assembly kinetics are monitored by ThT fluorescence at 482 nm (excited at 450 nm). Similar experiments are performed using maleimide-activated microtiter plates (Pierce) that were coated with CsgA 20 mer peptides. Briefly, 20 mer peptides containing an N-terminal cysteine and short PEG spacer (MW 0.39 kD) are dissolved in DMSO and incubated in maleimide-functionalized microtiter plates overnight at 100 μM (10% DMSO, 5.4 M GuHCl, 90 mM potassium phosphate, pH 7.2). The wells are washed extensively with reaction buffer, blocked with 3% BSA for several hours and unseeded reactions are conducted as described above. Seeded reactions are performed with unblocked microtiter plates without mixing beads, and the plates are typically mixed for 3 sec/min. The seeding kinetics are monitored by both ThT fluorescence (once per minute) and SDS resistance (endpoint) for approximately 45 minutes.

Production of CsgA Polypeptide

The gene encoding CsgA (accession number AAC74126) was cloned into a His-tagged overexpression construct and purified. The protocol was as follows:

1. CELLS EXPRESSING CsgA ARE RESUSPENDED IN 6M GdmCl (pH 7.4).

2. SAMPLES ARE CENTRIFUGED AT 3000 RPM FOR 30 MIN.

3. THE SUPERNATENT IS THEN ADDED TO PREWASHED NICKEL NTA RESIN (4 ML PER ML OF BEAD). THE SUSPENSION IS LEFT OVER NIGHT ON A ROCKER.

4. THE AGAROSE BEADS ARE THEN LOADED ON TO AN EMPTY COLUMN AND WASHED WITH 8M UREA (100 ML). BOUND CsgA IS ELUTED USING 6M GdmCl pH 2.0. THE PURIFIED PROTEIN IS METHANOL PRECIPITATED AND REDISSOLVED IN 90% FORMIC ACID OR HFIP FOR USE.

Additional details of methods and materials that may be used in certain aspects of the present invention are found in PCT application PCT/US2007/019910, in Krishnan and Lindquist, 2005, Nature, 435; 765-72, and Tessier and Lindquist, 2007, Nature, 447; 556-561.

### Example 1

### Identification of Peptides that Mediate Curli Formation

It was hypothesized that short peptides could be used to identify important domains within the CsgB sequences responsible for nucleating assembly of curli. A library of overlapping peptides was synthesized with 20 residues at their C-terminus derived from CsgB, a PEG spacer and an N-terminal, double lysine tag for covalent immobilization. Denatured peptides were arrayed on reactive glass slides and their interaction with soluble, fluorescently labeled CsgA was studied.

Sequence accession numbers for CsgB and CsgA used in this Example are: AAC74125 and AAC74126, respectively. A peptide library of CsgB sequences was generated and immobilized on a glass slide essentially as described above. The peptides (most of which were 20 amino acids in length) were staggered by 2 amino acids across the CsgB sequence. Upon incubating the arrayed peptide library essentially as described above with low concentrations of recombinantly expressed CsgA (˜2 μm), of which about 10% was labeled with ALEXA-647, we found 3 sequences (2 independent sequences) in CsgB that nucleated CsgA (See FIG. 8). Notably, the arrays also contained a full complement of CsgA peptides, but none of these induced assembly of CsgA. It will be understood that binding typically depends on the peptide concentration, CsgA concentration, and stringency of wash. Conditions used in this experiment detect peptides that have very high affinity for soluble CsgA. Using a less stringent wash or higher CsgA concentrations (e.g., 5-fold higher), would identify more peptides.

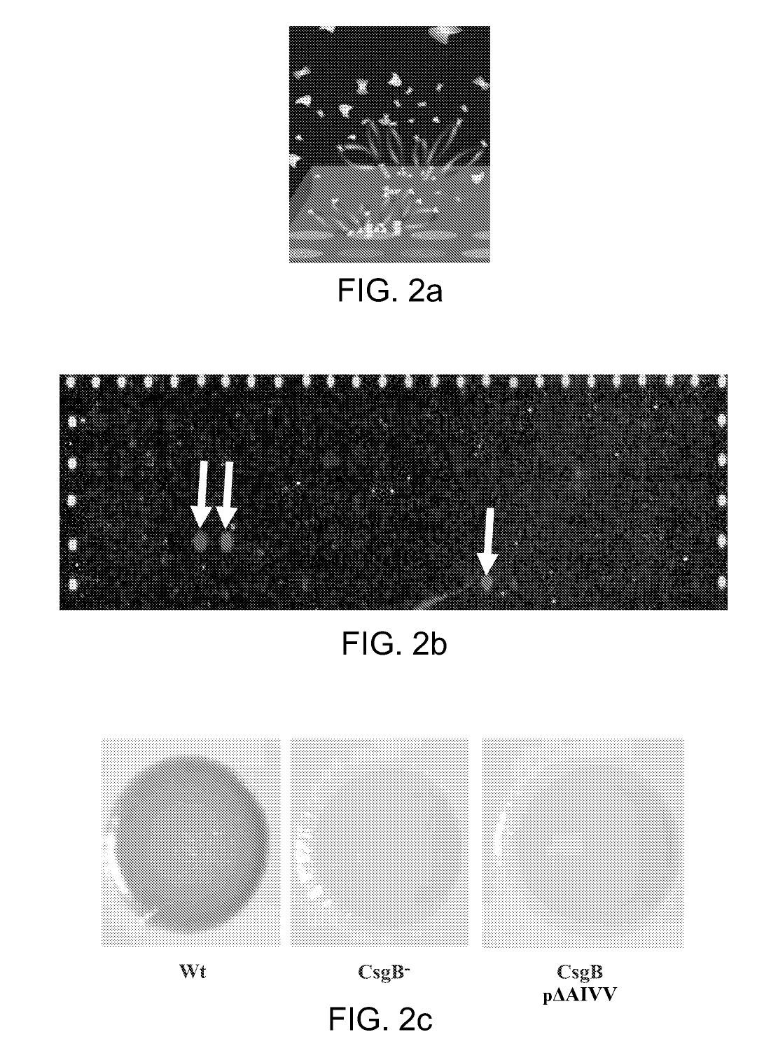

To provide conclusive evidence that the sequences identified in CsgB by the peptide array were indeed responsible for nucleating CsgA, a stretch of 4 amino acids within the nucleating sequence were removed and the mutated gene re-incorporated into bacterial cells by recombinant DNA engineering. Bacterial cells harboring this small deletion completely lost their ability to form functional curli (FIG. 2C).

### Example 2

### Structure-Activity Analysis of Variant CsgB Peptide Sequences

Variants of the CsgB peptides identified as described in Example 1 are synthesized, in which one or more individual amino acid(s) is/are replaced by a different amino acid. The peptides are attached to a support and contacted with soluble CsgA polypeptide. Seeding rates for the variants relative to wild type peptides is determined. Variants showing higher seeding ability are identified.

### Example 3

### Structure-Activity Analysis of Variant CsgB Peptide Sequences

Variants of the CsgB peptides identified as described in Example 1 are synthesized, in which one or more individual amino acid(s) is/are replaced by a different amino acid. A Wild type CsgB peptides identified as described in Example 1 are attached to a support and contacted with soluble CsgA polypeptide in the presence of excess variant peptide. The ability of a variant to inhibit aggregate formation is assessed. Variants that are able to effectively reduce the rate of aggregate formation are identified.

While the invention has been particularly shown and described with reference to specific embodiments, it should be understood by those skilled in the art that various changes in form and detail may be made therein without departing from the spirit and scope of the invention as defined by the appended claims. Those skilled in the art will recognize, or be able to ascertain using no more than routine experimentation, many equivalents to the specific embodiments of the invention described herein.

The articles “a”, “an”, and “the” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims or from the description is introduced into another claim dependent on the same base claim (or, as relevant, any other claim) unless otherwise indicated or unless it would be evident to one of ordinary skill in the art that a contradiction or inconsistency would arise. Where elements are presented as lists, e.g., in Markush group or similar format, it is to be understood that each subgroup of the elements is also disclosed, and any element(s) can be removed from the group. It should it be understood that, in general, where the invention, or aspects of the invention, is/are referred to as comprising particular elements, features, etc., certain embodiments of the invention or aspects of the invention consist, or consist essentially of, such elements, features, etc. It should also be understood that any embodiment of the invention can be explicitly excluded from the claims, regardless of whether the specific exclusion is recited in the specification. Aspects of the invention are described herein with particular reference to curli fibers and polypeptides that comprise curli fibers, but it should be understood that the invention encompasses embodiments that relate to other polypeptides that form heteroaggregates.

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, can be values expressed as percentages. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”.

Unless clearly indicated to the contrary, in any methods claimed herein that include more than one act, the order of the acts of the method is not necessarily limited to the order in which the acts of the method are recited, but the invention includes embodiments in which the order is so limited. It should also be understood that where compositions are claimed or described herein, the invention also provides methods of using and making the compositions, and where methods are claimed or described herein, the invention also provides compositions made according to the claimed methods.

1. A peptide between 5 and 50 amino acid long whose sequence comprises at least 5 and no more than 50 contiguous amino acids of the sequence of a first amyloidogenic polypeptide, wherein the peptide is capable of nucleating amyloid formation by a second amyloidogenic polypeptide.

2.-4. (canceled)

5. The peptide of claim 1, wherein at least one of the amyloidogenic polypeptides is a component of a biofilm generated by a bacterium.

6.-9. (canceled)

10. The peptide of claim 5, wherein the bacterium is a member of a genus selected from Escherichia, Klebsiella, Salmonella, and Shigella.

11. The peptide of claim 1, wherein the first amyloidogenic polypeptide is a CsgB polypeptide.

12. The peptide of claim 1, wherein the first and second amyloidogenic polypeptides are a CsgB polypeptide and a CsgA polypeptide, respectively.

13.-27. (canceled)

28. A composition comprising the peptide of claim 1.

29. The composition of claim 28, further comprising a second amyloidogenic polypeptide capable of forming an amyloid when incubated with the peptide under suitable conditions.

30.-35. (canceled)

36. The composition of claim 28, further comprising a test agent.

37. (canceled)

38. A collection comprising at least 10 different peptides, wherein the peptides are between 6 and 50 amino acid in length and have a sequence that comprises at least 8 and no more than 50 contiguous amino acids of a first amyloidogenic polypeptide, wherein the first amyloidogenic polypeptide is capable of nucleating amyloid formation by a second amyloidogenic polypeptide.

39.-45. (canceled)

46. The collection of claim 38, wherein at least one of the amyloidogenic polypeptides is a biofilm-forming polypeptide produced by a bacterium.

47.-50. (canceled)

51. The collection of claim 46, wherein the bacterium is a member of a genus selected from Escherichia, Klebsiella, Salmonella, and Shigella.

52. The collection of claim 38, wherein the first amyloidogenic polypeptide is a CsgB polypeptide.

53.-68. (canceled)

69. A method of identifying an aggregation domain of a first amyloidogenic polypeptide comprising the steps of:
(i) providing an array comprising a plurality of peptides, wherein the peptides are fragments of a first amyloidogenic polypeptide;
(ii) contacting the array with a second amyloidogenic polypeptide; and
(iii) identifying a peptide to which the second amyloidogenic polypeptide binds, thereby identifying an aggregation domain of the first amyloidogenic polypeptide.

70.-80. (canceled)

81. The method of claim 69, wherein at least one of the amyloidogenic polypeptides has a sequence at least 90% identical to a polypeptide component of a naturally occurring biofilm-forming polypeptide produced by a bacterium.

82.-90. (canceled)

91. A method of identifying an agent for modulating amyloid formation comprising:
(i) providing a composition comprising: (a) a peptide that is between 8 and 50 amino acid in length and has a sequence that comprises at least 8 and no more than 50 contiguous amino acids of a first amyloidogenic polypeptide; (b) a second amyloidogenic polypeptide; and (c) a test agent, wherein the peptide is capable of binding to the second amyloidogenic polypeptide in the absence of the test agent; and (ii) identifying the test agent as an agent for modulating amyloid formation if presence of the test agent alters the extent or rate of binding of the peptide and the polypeptide.

92.-95. (canceled)

96. The method of claim 91, wherein the peptide is attached to a support.

97. The method of claim 91, wherein step (ii) comprises identifying the agent as an inhibitor of amyloid formation or maintenance if the presence of the test agent reduces the extent or rate of binding of the peptide and the polypeptide.

98. The method of claim 91, wherein the composition further comprises an indicator of amyloid formation.

99. The method of claim 91, wherein at least one of the amyloidogenic polypeptides polypeptides has a sequence at least 90% identical to a polypeptide component of a naturally occurring biofilm-forming polypeptide produced by a bacterium.

100.-103. (canceled)

104. The method of claim 99, wherein the bacterium is a member of a genus selected from Escherichia, Klebsiella, Salmonella, and Shigella.

105.-122. (canceled)
